(19) United States
(12) Patent Application Publication
(10) Pub. No.: US 2025/0052756 A1
(43) Pub. Date: Feb. 13, 2025

(54) METHODS FOR DETERMINING THE PROGNOSIS AND STAGE OF A DISEASE OR DISORDER

(71) Applicant: HESSIAN LABS, INC., Worcester, MA (US)

(72) Inventors: Giannoula Lakka KLEMENT, Boston, MA (US); Abdo ABOU-SLAYBI, Worcester, MA (US); Darrol G. ROBERTS, Worcester, MA (US)

(73) Assignee: HESSIAN LABS, INC., Worcester, MA (US)

(21) Appl. No.: 18/718,931

(22) PCT Filed: Dec. 13, 2022
(86) PCT No.: PCT/US2022/052628

§ 371 Date: Jun. 12, 2024

Related U.S. Application Data
(60) Provisional application No. 63/288,775, filed on Dec. 13, 2021.

(51) Int. Cl. G01N 33/574; G01N 33/543

(52) U.S. Cl. CPC G01N 33/57434; G01N 33/54386

(57) ABSTRACT

Eleven percent of all men will develop prostate cancer (PC). In the US, 3 million men are living with the disease, 192,000 new cases are expected, and 33,000 men will die from the illness this year. PC is the second leading cause of cancer (CA) deaths in men worldwide, the diagnosis of PC relies on a combination of tests and procedures, including the digital rectal exam (DRE), magnetic resonance imaging (MRI), ultrasound, and multiple tissue biopsies, each with escalating costs and risks. In 2018, the cost of PC care in the US was $15.3 billion dollars, of which $5.7 billion was spent on diagnosis and $8.3 billion on management. There is a critical need for an early, accurate, reliable and cancer specific diagnostic for PC. The technology as described herein relates to compositions and methods for diagnosing and staging of cancer in a subject.

### CROSS REFERENCE TO RELATED APPLICATIONS

This application claims benefit under 35 U.S.C. § 119(e) of U.S. Provisional Application No. 63/288,775, filed Dec. 13, 2021, the contents of which are incorporated herein by reference in its entirety.

### TECHNICAL FIELD

The field of the invention relates to the detection and staging of cancer.

### BACKGROUND

Eleven percent of all men will develop prostate cancer (PC). In the US, 3 million men are living with the disease, 192,000 new cases are expected, and 33,000 men will die from the illness this year. PC is the second leading cause of cancer (CA) deaths in men worldwide. The prostate-specific antigen (PSA) test, presently the gold standard, was developed and instituted to ensure early diagnosis. However, it measures an antigen from normal prostate epithelial cells rather than cancer-specific cells, and as such its levels are unreliable for predicting cancer progression. In prostate hypertrophy, PSA levels increase without malignant transformation, producing “false positives” in up to 75% of cases. The confirmatory tests come with risk of treatment complications, such as lifelong erectile dysfunction and incontinence. Yet, the diagnosis of PC relies on a combination of tests and procedures, including the digital rectal exam (DRE), magnetic resonance imaging (MRI), ultrasound, and multiple tissue biopsies, each with escalating costs and risks. In 2018, the cost of PC care in the US was $15.3 billion dollars, of which $5.7 billion was spent on diagnosis and $8.3 billion on management. There is a critical need for an early, accurate, reliable and cancer specific diagnostic for PC.

### SUMMARY

Provided herein are methods and compositions related to the use of platelet-sequestered proteins for the diagnosis of cancer.

Accordingly, provided herein in one aspect is a method of diagnosing cancer in a subject, the method comprising: (a) measuring, in a sample of platelets from the subject, the levels of a set of peptides or polypeptides comprising subsets of polypeptides representing functions in cancer development including each of malignant cell transformation, invasion, angiogenesis and metastasis; (b) comparing the levels of the set of polypeptides measured in step (a) with the levels of the same set of polypeptides in a reference preparation of platelets, plasma, circulating cells or to a reference value or range thereof for each polypeptide; wherein a difference in the level of polypeptides representative of one or more of the functions relative to the reference is indicative of cancer status in the subject.

In one embodiment of this aspect and all other aspects provided herein, the set of polypeptides comprises a plurality of polypeptides associated with each function.

In another embodiment of this aspect and all other aspects provided herein, the polypeptides representative of functions in cancer development include cancer stimulatory and/or cancer inhibitory polypeptides.

In another embodiment of this aspect and all other aspects provided herein, an increase in one or more cancer stimulatory polypeptides or a decrease in one or more cancer inhibitory polypeptides is indicative of cancer progression.

In another embodiment of this aspect and all other aspects provided herein, a decrease in one or more cancer stimulatory polypeptides or an increase in one or more cancer inhibitory polypeptides is indicative of cancer regression or therapy responsiveness.

In another embodiment of this aspect and all other aspects provided herein, the method diagnoses prostate cancer, and the polypeptides representative of function in malignant cell transformation include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

In another embodiment of this aspect and all other aspects provided herein, the method diagnoses prostate cancer, and the polypeptides representative of functions in malignant cell transformation include two or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

In another embodiment of this aspect and all other aspects provided herein, the method diagnoses prostate cancer, and the polypeptides representative of functions in malignant cell transformation include each of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

In another embodiment of this aspect and all other aspects provided herein, the method diagnoses prostate cancer, and the polypeptides representative of function in cancer cell invasion (e.g., local tissue cancer invasion) include one or more of MYCBP2, CD47, FKBP1A, and Tribbles homolog 2.

In another embodiment of this aspect and all other aspects provided herein, the method diagnoses prostate cancer, and the polypeptides representative of function in cancer cell invasion (e.g., local tissue cancer invasion) include two or more of MYCBP2, CD47, FKBP1A, and Tribbles homolog 2.

In another embodiment of this aspect and all other aspects provided herein, the method diagnoses prostate cancer, and the polypeptides representative of function in angiogenesis include one or more of PF4, TSP-1, basic FGF, VEGF, PDGF beta, and Integrin beta-3.

In another embodiment of this aspect and all other aspects provided herein, the method diagnoses prostate cancer, and the polypeptides representative of function in angiogenesis include two or more of PF4, TSP-1, basic FGF, VEGF, PDGF beta, and Integrin beta-3.

In another embodiment of this aspect and all other aspects provided herein, the cancer is prostate cancer, and the polypeptides representative of function in angiogenesis include each of PF4, TSP-1, and Integrin beta-3 and optionally basic FGF, VEGF, and PDGF beta.

In another embodiment of this aspect and all other aspects provided herein, the method diagnoses prostate cancer, and the polypeptides representative of function in metastasis include one or more of CAMSAP2, SRGN, RANK-L, TGF-beta, IGF-1, and Synaptobrevin homolog YKT6.

In another embodiment of this aspect and all other aspects provided herein, the method diagnoses prostate cancer, and the polypeptides representative of function in metastasis include two or more of CAMSAP2, SRGN, RANK-L, TGF-beta, IGF-1, and Synaptobrevin homolog YKT6.

In another embodiment of this aspect and all other aspects provided herein, the method diagnoses prostate cancer, and the polypeptides representative of function in metastasis include each of CAMSAP2, SRGN, and Synaptobrevin homolog YKT6 and optionally one or more of RANK-L, TGF-beta, and/or IGF-1.

In another embodiment of this aspect and all other aspects provided herein, the aggressiveness or stage of a cancer is indicated by the presence of markers in a particular functional class of polypeptides (e.g., polypeptides for malignant cell transformation, invasion, angiogenesis and/or metastasis) that fall within a reference range for subjects having a particular stage of cancer.

In another embodiment of this aspect and all other aspects provided herein, relative aggressiveness increases as markers from the functional classes are found to vary, in the order of polypeptides for malignant cell transformation, polypeptides for invasion, polypeptides for angiogenesis, and polypeptides for metastasis.

In another embodiment of this aspect and all other aspects provided herein, the method further comprises administering an anti-cancer agent when a difference in the level of polypeptides representative of one or more of the functions relative to the reference indicates cancer in the subject.

In another embodiment of this aspect and all other aspects provided herein, cancer progression is indicated when the balance of cancer stimulatory vs cancer inhibitory polypeptides is disrupted such that cancer stimulatory polypeptides are increased and/or cancer inhibitory polypeptides are decreased relative to the reference (e.g., platelets from a subject(s) lacking cancer).

Also provided herein, in another aspect, is a device for the diagnosis of cancer or of cancer progression, the device comprising reagents sufficient to detect, in a sample of platelets or platelet proteins from a subject, the presence and/or amount of a set of polypeptides comprising subsets of polypeptides that are representative of functions in cancer development including each of malignant cell transformation, invasion, angiogenesis and metastasis.

In another embodiment of this aspect and all other aspects provided herein, the set of polypeptides comprises a plurality of polypeptides associated with each function.

In another embodiment of this aspect and all other aspects provided herein, the polypeptides representative of functions in cancer development include cancer stimulatory and cancer inhibitory polypeptides.

In another embodiment of this aspect and all other aspects provided herein, the polypeptides representative of function in malignant cell transformation include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

In another embodiment of this aspect and all other aspects provided herein, the polypeptides representative of functions in malignant cell transformation include two or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

In another embodiment of this aspect and all other aspects provided herein, the polypeptides representative of functions in malignant cell transformation include each of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

In another embodiment of this aspect and all other aspects provided herein, the polypeptides representative of function in cancer cell invasion (e.g., local tissue invasion) include one or more of MYCBP2, CD47, FKBP1A, and Tribbles homolog 2.

In another embodiment of this aspect and all other aspects provided herein, the polypeptides representative of function in cancer cell invasion (e.g., local tissue invasion) include two or more of MYCBP2, CD47, FKBP1A, and Tribbles homolog 2.

In another embodiment of this aspect and all other aspects provided herein, the polypeptides representative of function in angiogenesis include one or more of PF4, TSP-1, basic FGF, VEGF, PDGF beta, and Integrin beta-3.

In another embodiment of this aspect and all other aspects provided herein, the polypeptides representative of function in angiogenesis include two or more of PF4, TSP-1, basic FGF, VEGF, PDGF beta, and Integrin beta-3.

In another embodiment of this aspect and all other aspects provided herein, the polypeptides representative of function in angiogenesis include each of PF4, TSP-1, basic FGF, VEGF, PDGF beta, and Integrin beta-3.

In another embodiment of this aspect and all other aspects provided herein, the polypeptides representative of function in metastasis include one or more of CAMSAP2, SRGN, RANK-L, TGF-beta, IGF-1, and Synaptobrevin homolog YKT6.

In another embodiment of this aspect and all other aspects provided herein, the polypeptides representative of function in metastasis include two or more of CAMSAP2, SRGN, RANK-L, TGF-beta, IGF-1, and Synaptobrevin homolog YKT6.

In another embodiment of this aspect and all other aspects provided herein, the polypeptides representative of function in metastasis include each of CAMSAP2, SRGN, RANK-L, TGF-beta, IGF-1, and Synaptobrevin homolog YKT6.

In another embodiment of this aspect and all other aspects provided herein, the device comprises a solid substrate comprising reagents permitting detection of the presence and/or amount of the set of polypeptides.

In another embodiment of this aspect and all other aspects provided herein, the solid substrate comprises a lateral flow test strip, a microfluidics chamber, a dipstick, beads, or an enzyme-linked immunosorbent assay (ELISA).

In another embodiment of this aspect and all other aspects provided herein, the solid substrate is coated with a glucosaminoglycan.

Another aspect provided herein relates to a kit for the detection, in a sample of platelets or platelet proteins from a subject, of the presence and/or amount of a set of polypeptides comprising subsets of polypeptides that are representative of functions in cancer development including each of transformation, invasion, angiogenesis and metastasis, the kit comprising reagents sufficient to detect, in a sample of platelets or platelet proteins from the subject, the presence and/or amount of the set of polypeptides comprising subsets of polypeptides that are representative of functions in cancer development including each of transformation, invasion, angiogenesis and metastasis, and packaging materials therefor.

Also provided herein, in another aspect, is a kit for the staging of cancer, the kit comprising reagents necessary for detecting in a platelet sample the presence and/or amount of sets of polypeptides in functional cancer development categories including each of malignant cell transformation, invasion, angiogenesis and metastasis.

In another embodiment of this aspect and all other aspects provided herein, the kit comprises at least one solid support comprising reagents sufficient to detect the presence and/or amount of the sets of polypeptides.

In another embodiment of this aspect and all other aspects provided herein, the solid support comprises a lateral flow test strip, a microfluidics chamber, a dipstick, beads, or an enzyme-linked immunosorbent assay (ELISA).

In another embodiment of this aspect and all other aspects provided herein, the solid substrate is coated with a glucosaminoglycan.

In another embodiment of this aspect and all other aspects provided herein, the kit comprises reagents for the detection of actin.

In another embodiment of this aspect and all other aspects provided herein, the polypeptides representative of function in malignant cell transformation include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

In another embodiment of this aspect and all other aspects provided herein, the polypeptides representative of functions in malignant cell transformation include two or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

In another embodiment of this aspect and all other aspects provided herein, the polypeptides representative of functions in malignant cell transformation include each of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

In another embodiment of this aspect and all other aspects provided herein, the polypeptides representative of function in cancer cell invasion include one or more of MYCBP2, CD47, FKBP1A, and Tribbles homolog 2.

In another embodiment of this aspect and all other aspects provided herein, the polypeptides representative of function in cancer cell invasion include two or more of MYCBP2, CD47, FKBP1A, and Tribbles homolog 2.

In another embodiment of this aspect and all other aspects provided herein, the polypeptides representative of function in angiogenesis include one or more of PF4, TSP-1, basic FGF, VEGF, PDGF beta, and Integrin beta-3.

In another embodiment of this aspect and all other aspects provided herein, the polypeptides representative of function in angiogenesis include two or more of PF4, TSP-1, basic FGF, VEGF, PDGF beta, and Integrin beta-3.

In another embodiment of this aspect and all other aspects provided herein, the polypeptides representative of function in angiogenesis include each of PF4, TSP-1, basic FGF, VEGF, PDGF beta, and Integrin beta-3.

In another embodiment of this aspect and all other aspects provided herein, the polypeptides representative of function in metastasis include one or more of CAMSAP2, SRGN, RANK-L, TGF-beta, IGF-1, and Synaptobrevin homolog YKT6.

In another embodiment of this aspect and all other aspects provided herein, the polypeptides representative of function in metastasis include two or more of CAMSAP2, SRGN, RANK-L, TGF-beta, IGF-1, and Synaptobrevin homolog YKT6.

In another embodiment of this aspect and all other aspects provided herein, the polypeptides representative of function in metastasis include each of CAMSAP2, SRGN, and Synaptobrevin homolog YKT6 and optionally one or more of RANK-L, TGF-beta, and IGF-1.

Also provided herein, in another aspect is a solid support comprising reagents sufficient to detect, in a sample of platelet proteins from a subject, polypeptides in each of a set of functional cancer development categories including malignant cell transformation, invasion, angiogenesis and metastasis.

In one embodiment of this aspect and all other aspects provided herein, the reagents sufficient to detect the presence and/or amount of the sets of polypeptides are arranged on the support in four regions, one region each for reagents sufficient to detect polypeptides in functional cancer development categories including malignant cell transformation, invasion, angiogenesis and metastasis, wherein each region comprises reagents sufficient to detect the presence and/or amount of one or more polypeptides in one of the respective functional cancer development categories.

In another embodiment of this aspect and all other aspects provided herein, each region comprises pooled reagents sufficient to detect the presence of a plurality of polypeptides in the respective functional cancer development category.

In another embodiment of this aspect and all other aspects provided herein, the amount of detectable signal in each region upon detection of the sets of polypeptides in a sample of platelets or platelet proteins provides an indication of the presence and/or stage of cancer in the subject from whom the platelets were obtained.

Another aspect provided herein relates to a method of diagnosing or staging cancer in a subject, the method comprising contacting a solid support as described herein with a sample of platelets or platelet proteins obtained from the subject, wherein the contacting permits detection of the presence and/or amount of platelet proteins in the sample in one or more of the functional cancer development categories.

Another aspect provided herein relates to a kit comprising a solid support as described herein and packaging materials therefor.

Another aspect provided herein relates to a method of monitoring cancer treatment for efficacy in a subject, the method comprising: (a) measuring, in a first sample of platelets from the subject, the levels of a set of polypeptides comprising subsets of polypeptides that are representative of functions in cancer development including each of malignant cell transformation, invasion, angiogenesis and metastasis; (b) measuring, in a second sample of platelets from the subject, taken after administration of a cancer therapy to the subject following step (a), the levels of the set of polypeptides comprising subsets of polypeptides that are representative of functions in cancer development including each of malignant cell transformation, invasion, angiogenesis and metastasis; and (c) comparing the levels of the polypeptides measured in steps (a) and (b); wherein a change in the level of polypeptides in one or more of the subsets of polypeptides measured in steps (a) and (b) provides an indication of the efficacy of the cancer therapy.

In one embodiment of this aspect and all other aspects provided herein, the polypeptides representative of functions in cancer development include cancer stimulatory and cancer inhibitory polypeptides.

In another embodiment of this aspect and all other aspects provided herein, a decrease in the level of one or more cancer-stimulatory polypeptides, or an increase in the level of one or more cancer inhibitory polypeptides measured in step (b) relative to those measured in step (a) indicates that the cancer therapy is effective.

In another embodiment of this aspect and all other aspects provided herein, a positive prognostic change in the level of polypeptides representative of malignant cell transformation, invasion, angiogenesis or metastasis indicates the therapy is effective, and wherein a negative prognostic change in the level of polypeptides representative of malignant transformation, invasion, angiogenesis or metastasis indicates the therapy is not effective.

In another embodiment of this aspect and all other aspects provided herein, the method further comprises: when the comparing step indicates the therapy is effective, continuing to administer the therapy at the current dosage and frequency, or reducing the dosage and/or frequency of administration of the therapy; or when the comparing step indicates therapy is not effective, administering a different therapy alone or in conjunction with the therapy, or increasing the dosage and/or frequency of the therapy.

### BRIEF DESCRIPTION OF DRAWINGS

FIG. 1 is a schematic depicting an exemplary method for isolation of platelets and platelet-poor plasma.

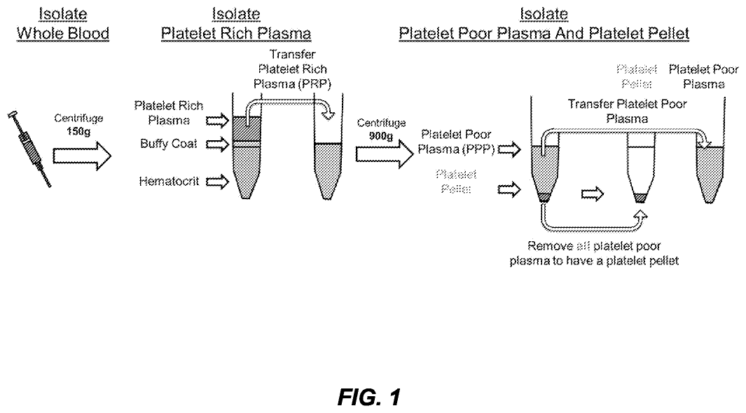

FIG. 2 is a schematic depicting an exemplary method for generating a profile of sequestered platelet proteins representative of cancer or cancer stages (e.g., a HessMap™).

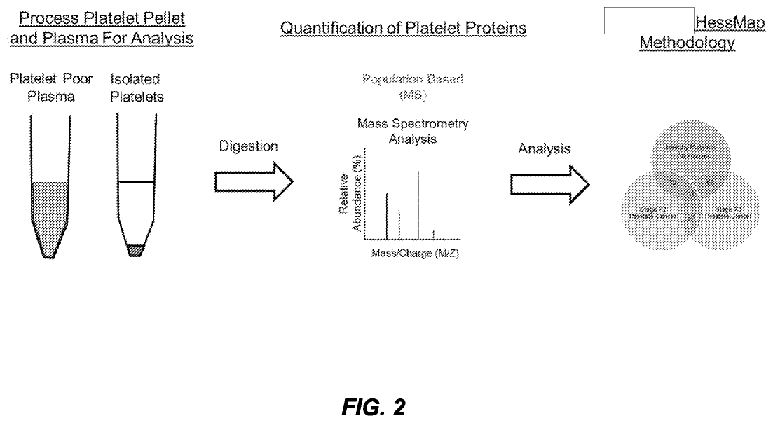

FIG. 3 is a table showing relative levels of cancer progression indicators in platelets. T2a=the tumor has invaded one-half (or less) of one side of the prostate. T3a=tumor has developed outside the prostate but has not spread to the seminal vesicles. The levels of stimulators increase from healthy to stage T2a and further in stage T3a, while the levels of inhibitors decrease from healthy to stage T2a an further in stage T3a.

FIG. 4 shows cancer stage specific proteins in both a box and whisker plot format.

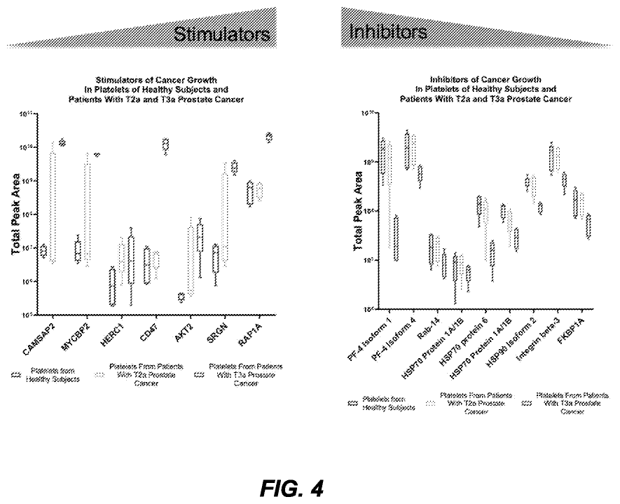

FIG. 5 is a table depicting normalized levels of cancer progression indicators in platelets. These proteins represent stimulators or inhibitors of carcinogenesis, cancer progression and metastasis. These data show that the levels of stimulators increase from healthy to stage T2a and further in stage T3a, while the levels of inhibitors decrease from healthy to stage T2a and further in stage T3a. These values are normalized to healthy platelet protein levels. These proteins can stage prostate cancer as being stage T2a or T3a.

FIG. 6 show normalized levels of cancer progression indicators in platelets.

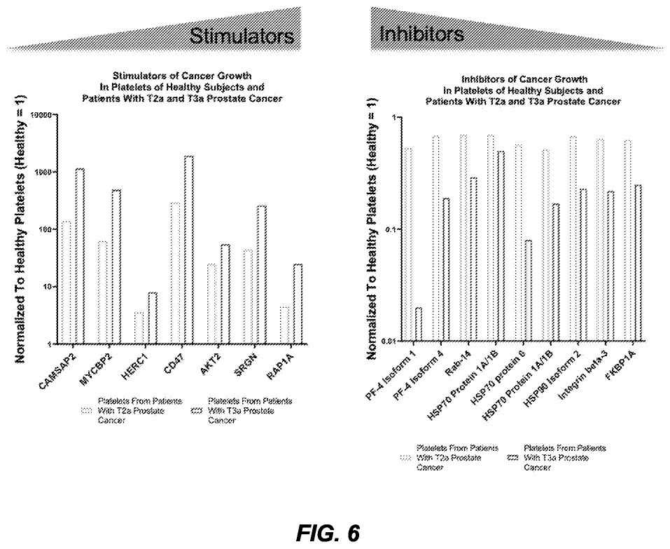

FIG. 7 is a table showing prostate cancer relevant biomarkers, i.e proteins or peptides not present in platelets of healthy subjects, but significantly elevated in platelets of subjects with prostate cancers. Average raw data values peak area times 10⁶. Overlap in the proteins such as HSP90 is due to differential expression of receptor subunits.

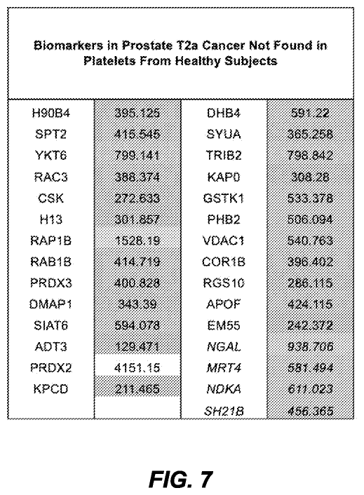

FIG. 8 is a graph showing prostate cancer polypeptides. A comparison of platelets isolated from 5 healthy subjects and 5 subjects with T2a prostate cancer revealed some proteins not detected in platelets from healthy controls but in subjects with stage T2a prostate cancer. Proteins were not detectable in healthy platelets or were present in miniscule amounts.

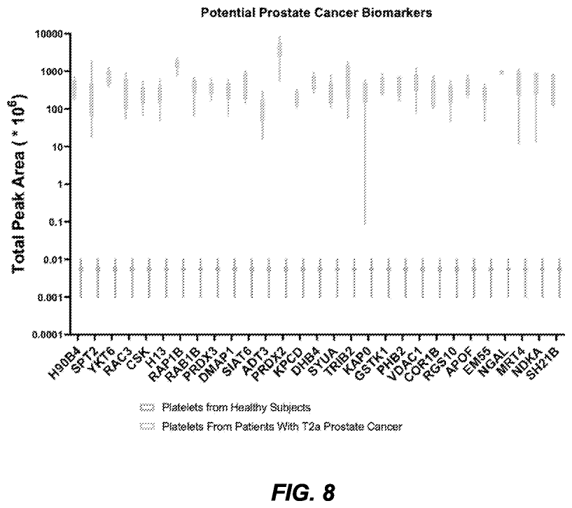

FIG. 9 An exemplary HessMap™ exposes proteins relevant to the identification of ‘escape from dormancy’. These data show how the sequestration of basic fibroblast growth factor (bFGF) in platelets can be an early marker of tumor becoming aggressive (i.e. signal an escape from dormancy). This dormant clone of human liposarcoma is known to require an average of 131 days to become palpable, i.e. reach a tumor size of 200-300 mm3. In this study, the platelet-associated bFGF detected the escape from dormancy in 2/5 mice at 32 days and in all mice by 120 days. The plasma bFGF in contrast, was not detectable in any of the mice throughout the 130 days of the study.

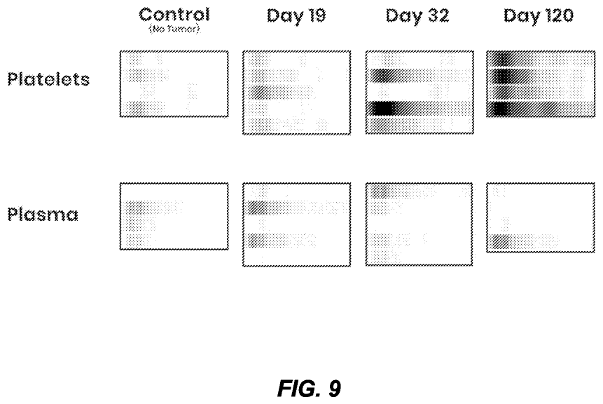

### DETAILED DESCRIPTION

Provided herein are methods, assays, and devices utilizing the presence of polypeptides in platelets or platelet preparations that are associated with, and are therefore predictive or indicative of disease states, such as cancer. Such platelet-sequestered polypeptides are indicative of the entire cancer-relevant protein landscape (e.g., detect the presence of cancer or permit cancer staging) and permits real-time evaluation of the efficacy and patient's response to therapy. Real-time evaluation of proteins in platelets can be used to guide patient treatment, as well as clinical trial enrollment and design. Polypeptides sequestered by platelets can be grouped based on their function and impact on cancer (e.g., angiogenesis, cell invasion/local tissue cancer invasion, metastasis etc.)

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the term “subject” includes humans and mammals. The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears.

In some embodiments, a mammal is a human. A subject can be of any age including a neonate toddler, child, teen, adult or a geriatric subject. Subjects that can be treated using the methods, and compositions as described herein include “healthy” individuals in respect to cancer, such as, e.g., (normal with no history of cancer; cancer not yet detected) but may have underlying health concerns, such as, e.g. (obesity, diabodies), and those suffering from, or at risk for, hyperplasia and/or cancer and/or associated inflammation. The subjects include human patients (adults and children) who have or are at risk of developing cancer.

A subject to be diagnosed, staged or treated according to the methods described herein can be a healthy individual or one who has been diagnosed with a disease, or condition associated with “neoplasia,” “cancer,” and/or inflammation, such as a disease or condition of the prostate, or one at risk of developing these conditions. A “healthy” subject is one that lacks detectable disease at the time of testing. A healthy individual, can also refer to an individual who has successfully been treated for neoplasia, and/or cancer, and is “disease free.”

As used herein, the term “cancer” refers to the presence of cells in tissue, or circulation, possessing characteristics typical of cancer-causing cells as understood by those of skill in the art of medicine. Exemplary characteristics of cancerous cells or tumor cells include, but are not limited to, uncontrolled cell proliferation, loss of normal functions, immortality, metastatic potential, lack of apoptotic activity or increase in anti-apoptotic activity, rapid growth and proliferation rate, unresponsiveness to regulatory mechanisms, and certain characteristic morphology and cellular markers.

As used herein, the terms “neoplasia,” and “hyperplasia,” are meant as disease that is caused by, or results from, high levels of cell division, or low levels of apoptosis, or both. Neoplasia is characterized by the pathological proliferation and migration to, or invasion of, other tissues or organs.

As used herein, the term “tumor,” refers to any neoplastic cell proliferation, whether malignant, or benign, and all pre-cancerous, and cancerous cells, and tissues including tissue resident, circulating and metastatic tumors.

As used herein, the term “isolated” refers to materials that are enriched, or removed from the native or endogenous state from which they are found. “Isolate” denotes a degree of separation from original source or surroundings. A related term, “Purify” denotes a degree of separation that is greater than isolation, such as e.g., (isolation of platelets from whole blood, and purification of platelet associated proteins). A “purified,” or “biologically pure” protein, is sufficiently free of other materials so that any impurities do not impact biological properties, or if they do it is to a negligible degree.

As used herein, the terms “platelet-associated protein” and “platelet-sequestered polypeptide” are synonymous and used to refer to short peptides, polypeptides or proteins that are detectable in isolated platelets and can be concentrated in platelets against a concentration gradient in plasma.

As used herein, the terms: “protein,” or “polypeptide,” or “peptide” is meant any chain of more than two natural or unnatural amino acids, regardless of post-translational modifications such as, e.g., (glycosylation or phosphorylation), constituting all or part of a naturally-occurring or non-naturally occurring polypeptide or peptide, as is described herein.

Unless otherwise defined, as used herein, the term “isolated polypeptide” is meant a polypeptide that has been separated from its natural components. An isolated polypeptide can be obtained, for example, by extraction from a natural source, such as whole blood or platelets.

As used herein, the term “polypeptides representative of functions in cancer development” refers to polypeptides that have been determined to be present or to be present in increased concentrations or occur in altered amounts during a particular phase of cancer development and their presence/absence or amount is therefore predictive or indicative of a particular disease state or stage of cancer.

As used herein, the term “functions in cancer development” refers to the multistep progression during cancer development and provides organizing principles for assigning platelet-associated polypeptides to a cancer having certain characteristics. Exemplary functions include malignant cell transformation, promotion of angiogenesis, cancer cell invasion (e.g., local tissue invasion), metastasis, and the like.

The term “malignant cell transformation,” as used herein refers to the process by which a cell no longer responds to mechanisms regulating normal proliferation or cell death (e.g., via apoptosis). Such a cell(s) can undergo uncontrolled proliferation and growth processes that may ultimately lead to the generation of a cancer mass or tumor. In some embodiments, malignant cell transformation is associated with changes in cell morphology, for example, increased nuclear to cytoplasmic ratio, dysplasia, chromatin re-organization, chromosomal alterations and accumulation of mutations, and loss of contact inhibition.

The term “angiogenesis” refers to the developmentally abnormal production of new blood vessels (e.g., neovascularization) induced in response to oncogenic stimuli as the nutrient needs of cancer cells or tumors increase. Cancers generally attain, as part of their development, the ability to actively promote angiogenesis to satisfy supply demands for nutrients, inflammatory cells, stem cells and other cancer supporting cells.

As used herein, the term “cancer cell invasion” refers to a process by which a cancer cell extends and penetrates into a neighboring tissue. The term “cancer cell invasion” can also be referred to herein as “local tissue cancer invasion.”

As used herein, the term “metastasis” refers to the movement of cancer cells from a primary site through the circulatory system or lymphatic system to a site distinct from the primary site, thereby permitting adhesion of the metastatic cells at the new site and establishment of a secondary cancer site (i.e. metastasis).

As used herein, the term “cancer stimulatory polypeptides” refers to polypeptides whose expression is associated with cancer growth and metastasis.

As used herein, the term “cancer inhibitory polypeptides” refers to polypeptides whose lack of expression or insufficient expression is associated with cancer growth and metastasis.

As used herein, the term “normalization,” refers to a method of modifying quantitative measures (e.g., protein concentration), to minimize and/or mitigate the impact of factors which can prevent equivalent comparison, for example, minimizing the impact of highly variable baseline of a protein in a population or removing the impact of differing amounts of starting material. Exemplary methods of normalization include, but are not limited to, utilizing a ratio in platelet number, or volume, or platelet associated reference, or series of references, to a reference protein such as actin, albumin, or another other platelet associated protein, to generate a ratio or “normalization/correction factor,” to correct for equivalent comparison. For example, if a platelet protein concentration is 10× in sample A, and 1× in sample B, and the platelet number is 10× in sample A, and 1× in sample B, a correction factor of 10 will be applied to sample B. (A Platelet number (10×) divided by B platelet number (IC)=a normalization factor of 10) correcting for the disparity in starting number indicating there is no difference in protein concentration in sample A compared to sample B.

The terms “decrease”, “reduced”, “reduction”, or “inhibit” are all used herein to mean a decrease or lessening of a property, level, or other parameter (such as the amount of a platelet-associated polypeptide or a disease symptom) by a statistically significant amount. In some embodiments, “reduce,” “reduction” or “decrease” or “inhibit” typically means a decrease in expression or activity of a given platelet-associated polypeptide by at least 10% as compared to a reference expression or activity level for the same platelet-associated polypeptide and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. Rather, the term “complete inhibition” is used to refer to a 100% inhibition as compared to an appropriate reference level. A decrease in a symptom of a given cancer can be preferably down to a level accepted as within the range of normal for an individual without the cancer.

In some embodiments, a platelet-associated polypeptide may be “absent” or “below detectable levels” under certain conditions. For example, in conditions where cancer is not present, one or more cancer stimulatory polypeptides may be absent or below detectable levels. Alternatively, in conditions associated with cancer or cancer progression, one or more cancer inhibitory polypeptides may be absent or below detectable levels.

The terms “increased,” “increase” or “enhance” or “activate” are all used herein to generally mean an increase of a property, level, or other parameter (e.g., amount of a platelet-associated polypeptide) by a statistically significant amount; for the avoidance of any doubt, the terms “increased”, “increase” or “enhance” or “activate” means an increase in expression or activity of a given platelet-associated polypeptide in the panel by at least 10% as compared to a reference level (e.g., a platelet sample from one or more healthy individuals), for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level. In one embodiment, a reference level can be the amount of each member of the panel of proteins in a subject or population of subjects lacking cancer.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

### Cancer

The methods, assays, and devices described herein can be used to detect the presence of polypeptides in platelets that are associated with particular cancer functions or processes, which in turn can be used to diagnose or stage a particular cancer. Such cancer functions or processes, include malignant cell transformation, angiogenesis, cell invasion, metastasis, etc. The methods and assays can be used to detect the presence of any cancer and/or to determine a particular stage of that cancer.

Some non-limiting examples of cancer that can be diagnosed, staged and/or treated using the methods and compositions described herein include, but are not limited to, carcinoma, lymphoma, blastoma, sarcoma, and leukemia. Other exemplary cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments, the carcinoma or sarcoma includes, but is not limited to, carcinomas and sarcomas found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. The types of carcinomas include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma. The types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

In one embodiment of the methods and compositions described herein, the subject having the tumor, cancer or malignant condition is undergoing, or has undergone, treatment with a conventional cancer therapy. In some embodiments, the cancer therapy is chemotherapy, radiation therapy, immunotherapy, targeted therapy/biological therapy (e.g., CAR-T cells) or a combination thereof.

Prostate cancer: In various examples, the subject has or is at risk of developing, a disease or condition, associated with precancerous prostate lesions and/or prostate cancer, of various associated tissues of the prostate, such as, e.g., prostate tissue and/or associated lymph nodes. In some embodiments, a method of differentially diagnosing presence, and stage of prostate cancer, or preventing unnecessary intervention, or treatment, of a disease or condition characterized by precancerous lesions, or prostate cancer, in a subject, with the method including isolation, examination, or characterization of platelet-sequestered proteins. In some embodiments, the disease or condition is characterized by prostate, spleen, stomach, bladder, colon, colorectal, neuroendocrine or prostate hyperplasia, such as, e.g., Adenomatous polyps, benign prostatic hyperplasia (BPH), and/or inflammation. In some embodiments, the diagnosis and staging of hyperplasia and tumor lesions is determined by AJCC (American Joint Committee on Cancer) TNM system including but not limited to; clinical, or pathologic tumor category, such as, e.g., (cT1-4, a-c, or pT1-4, a-c), the N category of spreading to lymph nodes, such as, e.g., (NO, or N1), the M category of metastasis, such as, e.g., (M0, or M1), and any combination of the above categories. In some embodiments, the subject has or is at risk of developing morbidity due to treatment, or lack of treatment of tumor lesions by such as, e.g., PSA screening, digital rectal exam. In some embodiments, the subject has or is at risk for irreversible morbidity of prostate cancer, such as, e.g., erectile dysfunction, or incontinence. In some embodiments, the disease or condition is a precancerous lesion, sarcoma, or carcinoma, derived from, or a precursor of, cancerous lesions of the prostate, spleen, stomach, bladder, colon, colorectal, neuroendocrine tissues and/or inflammation in a disease or condition.

### Platelets

Platelets, (thrombocytes), are small, irregularly shaped clear cell structures (meaning “cells” that do not have a nucleus), that are 2-3 m in diameter, made from pseudopodial extensions of precursor bone marrow cells termed “megakaryocytes.” In a healthy individual, platelet counts range from 150,000 to 450,000, platelets per microliter of blood, thus an adult with 5 liters of blood has a total of 750000000, to 2250000000 platelets. Thrombocytosis is having greater than 450,000 platelets per uL of blood while thrombocytopenia is less than 150,000 platelets per uL. The main function of platelets is the maintenance of hemostasis, but they also store and deliver many proteins necessary for tissue repair and regeneration. The classical role of platelets is in thrombus formation when damage to the endothelium, such as a laceration leads to damage of blood vessels and release of blood. Conversely, improper thrombus formation leads to morbidity, such as in acute coronary thrombosis. The ability of platelets to store and transport proteins is utilized in cancer for storage and transport of cancer promoting growth factors. As shown previously, angiogenesis regulators are sequestered in platelets actively, and against the concentration gradient (Cervi D et al. 2008, Klement G L et al. 2009). Furthermore, angiogenesis regulator sequestration is selective (Klement G L et al. 2009).

These observations indicate that platelets are “first responders” to injury and sequester proteins in a dynamic manner that permits the diagnosis of disease. Platelets carry a highly responsive and continuously changing ‘cargo’ of protein stimulators and inhibitors of e.g., cancer or neoplasia. For example, platelets can carry stimulators and inhibitors of angiogenesis that play a role in new blood vessel development and tissue reconstruction (e.g., VEGF, bFGF, and PDGF. As tissue reconstruction advances, the amount and type of stimulators and inhibitors in the platelet cargo changes in a predictable manner. The constantly changing platelet protein landscape (or protein signature) permits early cancer diagnosis, detection of tissue response to tumor progression and invasion, detection of metastatic growth, and monitoring of response to therapeutic agents. Proteins sequestered in platelets can permit monitoring of an entire cancer-relevant protein landscape including known polypeptides. Such platelet-sequestered proteins can be used to groups platelet-associated proteins by their functions and impacts (e.g. angiogenesis modulating, inflammatory, growth promoting, etc.) and can show changes in the platelet protein landscape relevant to disease stage and progression. The methods and compositions described herein allow for real time evaluation of the efficacy and patient response to therapy guiding patient treatment and clinical trial enrollment and design.

Prior studies indicate that cancer-relevant growth factors are actively sequestered in platelets that occurs against a concentration gradient in plasma and is more robust and more specific than protein concentrations found in plasma or serum. The data described herein in the working examples demonstrates that sequestration of cancer-relevant proteins occurs early in malignant progression, but unambiguously reflects the cancer stage and response to therapy.

Methods for the isolation of platelets are known to those of skill in the art and are described in “Current Protocols in Immunology by F. M. Ausubel, R. Brent, R. E. Kingston, D. D. Moore, J. G. Seidman, K. Struhl and V. B. Chanda (Editors), John Wiley & Sons, 2004.”, incorporated herein by reference. For example, whole blood is collected from a donor into vacutainer containing sodium citrate. The whole blood is then centrifuged at low g-force to separate the platelet rich plasma in a first stage from the other components. In a second stage of the procedure, platelet rich plasma is separated into a fresh tube and platelet concentrate obtained by centrifuging platelets at higher speed. The platelet concentrate is then resuspended in a standard lysis buffer and associated proteins are isolated.

In some embodiments, isolation of platelets is by routine phlebotomy techniques (e.g., venipuncture) utilizing collection tubes containing anti-coagulants, such as, but not limited to, e.g., 3.2% Sodium citrate, Sodium or lithium heparin without gel, Potassium EDTA, Sodium fluoride, and sodium or potassium oxalate or a combination thereof, in various concentrations of the above mentioned compounds. It should be noted that when serum is created all coagulation factors (including platelets) are clotted (e.g., a serum tube is sometimes called “clot” tube). As such, it cannot be used for platelet isolation. Thus, platelet isolation must always be performed in the absence of activator gel. In some embodiments, platelets are isolated from whole blood, platelet rich plasma, or washed platelets. In some embodiments, the systemic administration, or isolation of platelets includes, intravenous injection, or infusion of platelets to subsequently, be isolated, for characterization, or analysis, of platelet sequestered proteins. In some embodiments, normalization of platelet-associated proteins is by the number of platelets, such as, e.g., counts obtained by manual counting, or flow cytometry. In some embodiments, normalization of platelet-associated proteins is by volume, such as, e.g., platelet volume, Mean platelet volume (MPV). In some embodiments, normalization of platelet-associated proteins is by an endogenous protein (e.g., tubulin or actin, the skeletal proteins of platelets), exogenous protein (e.g., a labeled heavy peptide control for quantification of a specific protein), or compound sequestered by platelets, such as, e.g., loading exogenous derivative, or modification, of a platelet sequestered protein. In some embodiments, normalization of platelet associated proteins is by a single, or a combination, of selectively sequestered platelet proteins, such as, e.g., sequestered proteins in Tables 1-5. In some embodiments, the subject is a human.

Polypeptides that are Representative of Functions in Cancer Development

Proteins sequestered by platelets can provide dynamic information regarding the status of a disease or disorder, such as cancer. Platelet-sequestered proteins provide a ubiquitous, constantly circulating, physiologically dynamic system that monitors disease development, and biological events involving genetic changes not only in cancer cells, but also in the subject's physiology. That is, such proteins can bind to and be released by platelets in a dynamic manner reflective of their expression levels and the subject's disease status. For example, as cancer progresses, proteins sequestered by platelets will be indicative of the cancerous state, including an increase in the amount of cancer stimulators and/or a decrease in the amount of cancer inhibitors. Polypeptides sequestered by platelets can be representative of any function known to be associated with cancer development or progression including, but not limited to, proliferation, evasion of growth suppressors, resisting cell death, enabling replicative immortality, inducing angiogenesis, activating invasion, and metastasis malignant cell transformation, invasion, angiogenesis and/or metastasis.

TABLE 1
Platelet Proteins That Change Between Normal and Stage T2 Prostate Cancer

| Gene Name | Gene Symbol |
|---|---|
| Tubulin Beta 2A | TUBB2A |
| Symplekin | SYMPK |
| Arachidonate 12-Lipoxygenase | ALOX12 |
| Biliverdin Reductase B (Flavin Reductase (NADPH)) | BLVRB |
| Histone Cluster 1 H1a | HIST1H1A |
| Gelsolin (Amyloidosis Finnish Type) | GSN |
| Coagulation Factor XIII A1 Polypeptide | F13A1 |
| Peptidylprolyl Isomerase B (Cyclophilin B) | PPIB |
| Apolipoprotein C-III | APOC3 |
| Apolipoprotein A-II | APOA2 |
| Inter-Alpha (Globulin) Inhibitor H2 | ITIH2 |
| ADP-Ribosylation-Like Factor 6 Interacting Protein 5 | ARL6IP5 |
| Nucleoside Phosphorylase | PNP |
| Alpha-2-HS-Glycoprotein | AHSG |
| Leucine-Rich Alpha-2-Glycoprotein 1 | LRG1 |
| Phosphofructokinase Platelet | PFKP |
| Tropomyosin 4 | TPM4 |
| Cysteine And Glycine-Rich Protein 1 | CSRP1 |
| Transthyretin | TTR |
| Peptidylprolyl Isomerase A (Cyclophilin A)-Like 4E; Peptidylprolyl Isomerase A | MTPN, LY6G6D, |
| (Cyclophilin A)-Like 4G; Peptidylprolyl Isomerase A (Cyclophilin A)-Like 4F; | LOC652797, |
| Peptidylprolyl Isomerase A (Cyclophilin A)- Like 4A; Peptidylprolyl Isomerase A | PLGLB2, |
| (Cyclophilin A)-Like 4C; Peptidylprolyl Isomerase A (Cyclophilin A)-Like 4B | PPIAL3, |
| | PPIAL4C |
| Serpin Peptidase Inhibitor Clade A (Alpha-1 Antiproteinase Antitrypsin) Member 1 | SERPINA1 |
| Apolipoprotein D | APOD |
| Plasminogen | PLG |
| POTE Ankyrin Domain Family Member F | POTEF |
| Transferrin | TF |
| Complement Factor H | CFH |
| C-Type Lectin Domain Family 3 Member B | CLEC3B |
| Fibrinogen Alpha Chain | FGA |
| Complement Factor I | CFI |
| Fermitin Family Homolog 3 (Drosophila) | FERMT3 |
| Serpin Peptidase Inhibitor Clade F (Alpha-2 Antiplasmin Pigment | SERPINF2 |
| Epithelium Derived Factor) Member 2 | |
| Keratin 2 | KRT2 |
| Apolipoprotein C-II | APOC2 |
| Apolipoprotein A-I | APOA1 |
| Vitronectin | VTN |
| Complement Component 4 Binding Protein Alpha | C4BPA |
| Group-Specific Component (Vitamin D Binding Protein) | GC |
| Thrombospondin 1 | THBS1 |
| Keratin 9 | KRT9 |
| Coagulation Factor II (Thrombin) | F2 |
| Cytochrome B5 Reductase 3 | CYB5R3 |
| Hypothetical LOC100129500; Apolipoprotein E | HSPD1P6, |
| | APOE |
| Serpin Peptidase Inhibitor Clade C (Antithrombin) Member 1 | SERPINC1 |
| Coronin Actin Binding Protein 1C | CORO1C |
| Complement Factor B | CFB |
| Alpha-1-B Glycoprotein | A1BG |
| Tubulin Alpha 1a | TUBA1A |
| H3 Histone Family 3B (H3.3B); H3 Histone Family 3A Pseudogene; H3 | AZGP1P1, |
| Histone Family 3A; Similar To H3 Histone Family 3B; Similar To Histone | CALM3, |
| H3.3B | DEFA1B, |
| | LOC100133042, |
| | LOC440926 |
| Transaldolase 1 | TALDO1 |
| Chloride Intracellular Channel 1 | CLIC1 |
| Complement Component 1 Q Subcomponent B Chain | C1QB |
| Glyceraldehyde-3-Phosphate Dehydrogenase-Like 6; Hypothetical | CALM3, |
| Protein LOC100133042; Glyceraldehyde-3-Phosphate Dehydrogenase | DEFA1B, |
| | LOC100133042 |
| Similar To Complement C3 Precursor; Complement Component 3; | APOE, EZR, |
| Hypothetical Protein LOC100133511 | LOC653879 |
| Complement Component 1 S Subcomponent | C1S |
| Plasminogen-Like B2; Plasminogen-Like B1 | LOC652797, |
| | PLGLB2 |
| Glycoprotein Ib (Platelet) Beta Polypeptide | GP1BB |
| SH3 Domain Binding Glutamic Acid-Rich Protein Like 2 | SH3BGRL2 |
| Tubulin Tyrosine Ligase-Like Family Member 3; Actin Related Protein 2/3 | TPI1, ARPC4 |
| Complex Subunit 4 20 kda | |
| Transgelin 2 | TAGLN2 |
| Coactosin-Like 1 (Dictyostelium) | COTL1 |
| Carbonic Anhydrase II | CA2 |
| Kininogen 1 | KNG1 |
| Actinin Alpha 4 | ACTN4 |
| Lactate Dehydrogenase B | LDHB |
| SH3 Domain Binding Glutamic Acid-Rich Protein Like 3 | SH3BGRL3 |
| Angiotensinogen (Serpin Peptidase Inhibitor Clade A Member 8) | AGT |
| Enolase 1 (Alpha) | ENO1 |
| Orosomucoid 1 | ORM1 |
| Myosin Heavy Chain 9 Non-Muscle | MYH9 |

TABLE 2
Platelet Proteins That Change Between Normal and Stage T3 Prostate Cancer

| Gene Name | Gene Symbol |
|---|---|
| Complement Factor H | CFH |
| Transthyretin | TTR |
| Orosomucoid 1 | ORM1 |
| Orosomucoid 2 | ORM2 |
| Enolase 1 (Alpha) | ENO1 |
| Apolipoprotein A-I | APOA1 |
| Apolipoprotein A-II | APOA2 |
| Coagulation Factor XIII A1 Polypeptide | F13A1 |
| Apolipoprotein D | APOD |
| Carbonic Anhydrase I | CA1 |
| Apolipoprotein C-III | |
| Gelsolin (Amyloidosis Finnish Type) | GSN |
| Albumin | ALB |
| Serpin Peptidase Inhibitor Clade D (Heparin Cofactor) Member 1 | SERPIND1 |
| Tropomyosin 4 | TPM4 |
| Fibrinogen Beta Chain | FGB |
| Serpin Peptidase Inhibitor Clade A (Alpha-1 Antiproteinase Antitrypsin) Member 3 | SERPINA3 |
| Plasminogen | PLG |
| Group-Specific Component (Vitamin D Binding Protein) | GC |
| Transaldolase 1 | TALDO1 |
| Immunoglobulin J Polypeptide Linker Protein For Immunoglobulin Alpha And Mu | IGJ |
| Polypeptides | |
| Alpha-2-HS-Glycoprotein | AHSG |
| Transferrin | TF |
| Serpin Peptidase Inhibitor Clade A (Alpha-1 Antiproteinase Antitrypsin) Member 1 | SERPINA1 |
| Chloride Intracellular Channel 1 | CLIC1 |
| ADP-Ribosylation-Like Factor 6 Interacting Protein 5 | ARL6IP5 |
| Tubulin Beta 2A | TUBB2A |
| Peptidylprolyl Isomerase B (Cyclophilin B) | PPIB |
| Vitronectin | VTN |
| Phosphofructokinase Platelet | PFKP |
| Fibrinogen Gamma Chain | FGG |
| Apolipoprotein A-IV | APOA4 |
| Fermitin Family Homolog 3 (Drosophila) | FERMT3 |
| Complement Factor B | CFB |
| Complement Factor H-Related 1 | CFHR1 |
| Apolipoprotein C-II | APOC2 |
| Serpin Peptidase Inhibitor Clade F (Alpha-2 Antiplasmin Pigment | SERPINF2 |
| Epithelium Derived Factor) Member 2 | |
| Complement Component 4 Binding Protein Alpha | C4BPA |
| Nucleoside Phosphorylase | PNP |
| Fibrinogen Alpha Chain | FGA |
| Heat Shock Protein 90 kda Alpha (Cytosolic) Class A Member 2; Heat Shock | LOC644063, |
| Protein 90 kda Alpha (Cytosolic) Class A Member 1 | HP, HSPB1, |
| | HSP90AA2 |
| Symplekin | SYMPK |
| Complement Component 1 R Subcomponent | C1R |
| Heat Shock 70 kda Protein 1-Like | HSPA1L |
| Cytochrome B5 Reductase 3 | CYB5R3 |
| Serpin Peptidase Inhibitor Clade C (Antithrombin) Member 1 | SERPINC1 |
| Complement Component 1 Q Subcomponent B Chain | C1QB |
| Keratin 9 | KRT9 |
| Peptidylprolyl Isomerase A (Cyclophilin A)-Like 4E; Peptidylprolyl Isomerase A | MTPN, |
| (Cyclophilin A)-Like 4G; Peptidylprolyl Isomerase A (Cyclophilin A)-Like 4F; | LY6G6D, |
| Peptidylprolyl Isomerase A (Cyclophilin A)- Like 4A; Peptidylprolyl Isomerase A | LOC652797, |
| (Cyclophilin A)-Like 4C; Peptidylprolyl Isomerase A (Cyclophilin A)-Like 4B | PLGLB2, |
| | PPIAL3, |
| | PPIAL4C |
| Inter-Alpha (Globulin) Inhibitor H4 (Plasma Kallikrein-Sensitive Glycoprotein) | ITIH4 |
| Complement Component 9 | C9 |
| Inter-Alpha (Globulin) Inhibitor H1 | ITIH1 |
| Arachidonate 12-Lipoxygenase | ALOX12 |
| Alpha-1-B Glycoprotein | A1BG |
| Thrombospondin 1 | THBS1 |
| Complement Component 1 S Subcomponent | C1S |
| Keratin 10 | KRT10 |
| POTE Ankyrin Domain Family Member F | POTEF |
| LIM And Senescent Cell Antigen-Like Domains 1 | LIMS1 |
| Glycoprotein Ib (Platelet) Beta Polypeptide | GP1BB |
| Complement Component 8 Beta Polypeptide | C8B |
| Apolipoprotein C-I | APOC1 |
| Lactate Dehydrogenase B | LDHB |
| Myosin Heavy Chain 9 Non-Muscle | MYH9 |
| Glucose-6-Phosphate Dehydrogenase | G6PD |
| Biliverdin Reductase B (Flavin Reductase (NADPH)) | BLVRB |
| Haptoglobin-Related Protein; Haptoglobin | HLA-C, HLA-C, |
| | LOC644063, HP |
| Ceruloplasmin (Ferroxidase) | CP |

TABLE 3
Platelet proteins that change between Stage T2 and Stage T3 prostate cancer

| Gene Name | Gene Symbol |
|---|---|
| Tubulin Beta 1 | TUBB1 |
| Hemopexin | HPX |
| Myosin Heavy Chain 9 Non-Muscle | MYH9 |
| Tropomyosin 4 | TPM4 |
| Pro-Platelet Basic Protein (Chemokine (C-X-C Motif) Ligand 7) | PPBP |
| Similar To Complement C3 Precursor; Complement Component 3; | APOE, EZR, |
| Hypothetical Protein LOC100133511 | LOC653879 |
| Serpin Peptidase Inhibitor Clade A (Alpha-1 Antiproteinase Antitrypsin) Member 1 | SERPINA1 |
| Transferrin | TF |
| Apolipoprotein H (Beta-2-Glycoprotein I) | APOH |
| Hemoglobin Delta | HBD |
| Complement Factor H | CFH |
| Vinculin | VCL |
| Apolipoprotein A-I | APOA1 |
| Thrombospondin 1 | THBS1 |
| CAP Adenylate Cyclase-Associated Protein 1 (Yeast) | CAP1 |
| Protein S (Alpha) | PROS1 |
| Serpin Peptidase Inhibitor Clade A (Alpha-1 Antiproteinase Antitrypsin) Member 3 | SERPINA3 |
| Pregnancy-Zone Protein | PZP |
| ATP Synthase H+ Transporting Mitochondrial F1 Complex Alpha Subunit 1 | ATP5A1 |
| Cardiac Muscle | |
| Fibrinogen Beta Chain | FGB |
| Integrin Alpha 2b (Platelet Glycoprotein lib Of lib/liia Complex Antigen CD41) | ITGA2B |
| Hemoglobin Beta | HBB |
| Carbonic Anhydrase I | CA1 |
| Transthyretin | TTR |
| Complement Factor H | CFH |
| Orosomucoid 1 | ORM1 |
| Fibrinogen Gamma Chain | FGG |
| Hypothetical Gene Supported By AF081484; NM_006082; Tubulin Alpha 1b | ARPC4, |
| | LOC399942 |
| Tubulin Alpha 1a | TUBA1A |
| Complement Component 4 Binding Protein Alpha | C4BPA |

TABLE 4
Protein domains most frequent in platelet-sequestered proteins

| EGF-Like Domain: calcium- | Vitamin K-dependent carboxylation/ |
|---|---|
| binding EGF domain | gamma-carboxyglutamic |
| Hemopexin; Somatomedin B | Laminin G domain |
| C-type lectin (CLEC) | Laminin G2 domain |
| lipocalins | Thioredoxin |
| Serpin | Cyclophilin type peptidyl-prolyl |
| | cis-trans isomerase/CLD |
| Heparan Sulfate | Galectin |
| Pentraxins | CUB domain |
| S-100 | Activin receptor |
| CD47 domain | Plexin cytoplasmic RasGAP domain |

TABLE 5
Exemplary panel of polypeptides for cancer diagnosis and staging

| Gene Name | Gene Symbol |
|---|---|
| Ras-Related Protein Rab-1B | RAB1B |
| Ras-Related Protein Rap-1A | RAP1A |
| Heat shock protein 70 kDa protein 1B | HSPA1B |
| Heat shock 70 kDa protein 6 | HSPA6 |
| Heat shock 70 kDa protein 1-like | HSPA1L |
| Heat shock protein HSP 90-alpha isoform 2 | HSP90AA2P |
| MYC Binding Protein 2 | MYCBP2 |
| CD47 molecule | CD47 |
| FKBP Prolyl Isomerase 1A | FKBP1A |
| Tribbles homolog 2 | TRIB2 |
| Platelet Factor 4 | PF4 |
| Thrombospondin 1 | TSP-1 |
| Integrin beta-3 | ITGB3 |
| Calmodulin Regulated Spectrin Associated | CAMSAP2 |
| Protein Family Member 2 | |
| Serglycin | SRGN |
| Synaptobrevin homolog YKT6 | YKT6 V-SNARE |
| | Homolog |

In one embodiment, polypeptides representative of function in malignant cell transformation are detected in platelets and include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2. In one embodiments, such polypeptides representative of function in malignant cell transformation can be used to detect and/or stage prostate cancer.

In another embodiment, the method can be used to diagnose cancer (e.g., prostate cancer), and the polypeptides representative of function in cancer cell invasion (e.g., local tissue cancer invasion) include one or more of MYCBP2, CD47, FKBP1A, and Tribbles homolog 2.

In another embodiment, the method can be used to diagnose cancer (e.g., prostate cancer), and the polypeptides representative of function in angiogenesis include one or more of PF4, TSP-1, basic FGF, VEGF, PDGF beta, and Integrin beta-3.

In another embodiment, the method can be used to diagnose cancer (e.g., prostate cancer), and the polypeptides representative of function in metastasis include one or more of CAMSAP2, SRGN, RANK-L, TGF-beta, IGF-1, and Synaptobrevin homolog YKT6.

In one embodiment, the method comprises detection of at least one polypeptide from each functional group consisting of: polypeptides representative of function in malignant cell transformation, cancer cell invasion (e.g., local tissue cancer invasion), angiogenesis, and metastasis.

In other embodiments, the method comprises detection of at least 1 polypeptide (e.g., at least 2, at least 3, at least 4, at least 5, or all 6 polypeptides) from polypeptides representative of function in malignant cell transformation in combination with at least one polypeptide (e.g. at least 2, 3, or 4) from at least one functional group consisting of: polypeptides representative of function in cancer cell invasion (e.g., local tissue cancer invasion), angiogenesis, and/or metastasis.

In other embodiments, the method comprises detection of at least one polypeptide (e.g., at least 2, at least 3, or all 4 polypeptides) from polypeptides representative of function in cancer cell invasion (e.g., local tissue cancer invasion) in combination with at least one polypeptide (e.g. at least 2, 3, 4, 5 or 6) from at least one functional group consisting of: polypeptides representative of function in malignant cell transformation, angiogenesis, and/or metastasis.

In other embodiments, the method comprises detection of at least one polypeptide (e.g., at least 2, or all 3 polypeptides) from a set of polypeptides representative of function in angiogenesis in combination with at least one polypeptide (e.g. at least 2, 3, 4, 5 or 6) from at least one functional group consisting of: polypeptides representative of function in malignant cell transformation, cancer cell invasion (e.g., local tissue cancer invasion), and/or metastasis.

In other embodiments, the method comprises detection of at least one polypeptide (e.g., at least 2, or all 3 polypeptides) from polypeptides representative of function in metastasis in combination with at least one polypeptide (e.g. at least 2, 3, 4, 5 or 6) from at least one functional group consisting of: polypeptides representative of function in malignant cell transformation, cancer cell invasion (e.g., local tissue cancer invasion), and/or angiogenesis.

In one embodiment, the method comprises detection of at least 3 proteins selected from the group consisting of: RAB1B, RAP1A, Heat shock protein 70 kDa protein 1B, Heat shock 70 kDa protein 6, Heat shock 70 kDa protein 1-like, Heat shock protein HSP 90-alpha isoform 2, MYCBP2, CD47, FKBP1A, Tribbles homolog 2, PF4, TSP-1, basic FGF, VEGF, PDGF beta, Integrin beta-3, CAMSAP2, SRGN, RANK-L, TGF-beta, IGF-1, and Synaptobrevin homolog YKT6. In other embodiments, the method comprises detection of at least 4, at least 5, at least 6, at least 7, at least 8, at least 9, at least 10, at least 11, at least 12, at least 13, at least 14, at least 15, or all 16 proteins selected from the group consisting of: RAB1B, RAP1A, Heat shock protein 70 kDa protein 1B, Heat shock 70 kDa protein 6, Heat shock 70 kDa protein 1-like, Heat shock protein HSP 90-alpha isoform 2, MYCBP2, CD47, FKBP1A, Tribbles homolog 2, PF4, TSP-1, basic FGF, VEGF, PDGF beta, Integrin beta-3, CAMSAP2, SRGN, RANK-L, TGF-beta, IGF-1, and Synaptobrevin homolog YKT6.

In other embodiments, the method comprises detection of at least 4, at least 5, at least 6, at least 7, at least 8, at least 9, at least 10, at least 11, at least 12, at least 13, at least 14, at least 15, or all 16 proteins consisting essentially of polypeptides selected from the group consisting of: RAB1B, RAP1A, Heat shock protein 70 kDa protein 1B, Heat shock 70 kDa protein 6, Heat shock 70 kDa protein 1-like, Heat shock protein HSP 90-alpha isoform 2, MYCBP2, CD47, FKBP1A, Tribbles homolog 2, PF4, TSP-1, basic FGF, VEGF, PDGF beta, Integrin beta-3, CAMSAP2, SRGN, RANK-L, TGF-beta, IGF-1, and Synaptobrevin homolog YKT6.

In certain embodiments, the polypeptides detected in platelets consist of: RAB1B, RAP1A, Heat shock protein 70 kDa protein 1B, Heat shock 70 kDa protein 6, Heat shock 70 kDa protein 1-like, Heat shock protein HSP 90-alpha isoform 2, MYCBP2, CD47, FKBP1A, Tribbles homolog 2, PF4, TSP-1, basic FGF, VEGF, PDGF beta, Integrin beta-3, CAMSAP2, SRGN, RANK-L, TGF-beta, IGF-1, and Synaptobrevin homolog YKT6.

Balance of Stimulatory Vs. Inhibitory Polypeptides

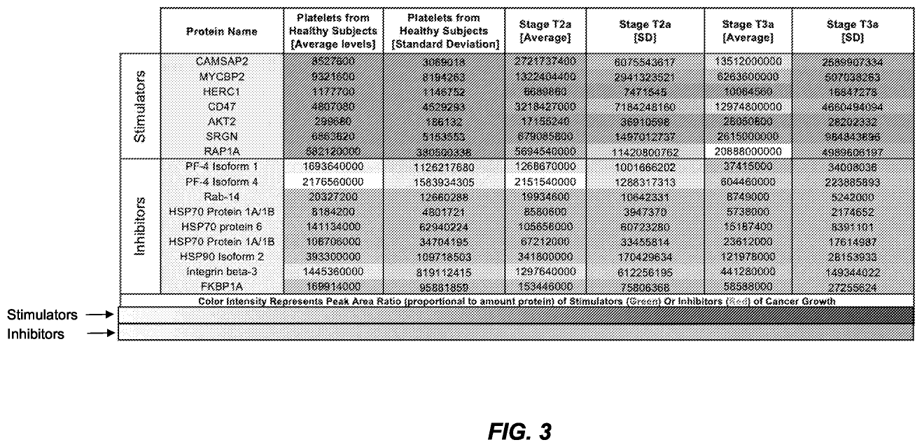

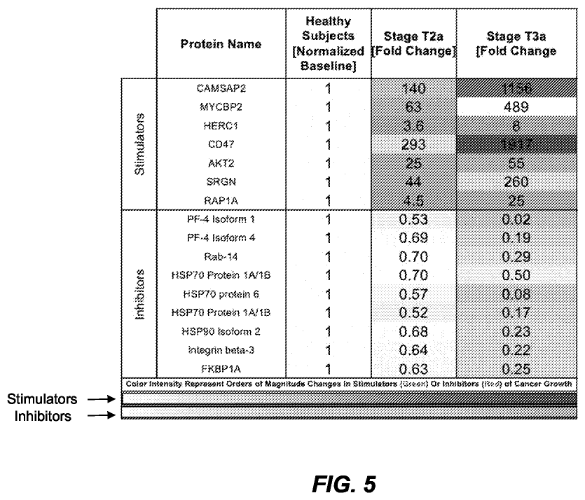

In certain embodiments, the polypeptides detected in platelets comprise at least one cancer stimulatory polypeptide and at least one cancer inhibitory polypeptide as depicted in FIG. 3 or 5. In one embodiment, at least 2, 3, 4, 5, 6 or all 7 of the cancer stimulatory polypeptides in FIG. 3 or 5 are detected in combination with at least one cancer inhibitory polypeptide as depicted in FIG. 3 or 5. In an alternative embodiment, at least 2, 3, 4, 5, 6 or all 7 of the cancer inhibitory polypeptides in FIG. 3 or 5 are detected in combination with at least one cancer stimulatory polypeptide as depicted in FIG. 3 or 5.

In one embodiment, at least two polypeptides (e.g., at least 3, 4, 5, 6, 7, 8, 9, 10, 11, 12, 13 or all 14) polypeptides selected from the group consisting of: CAMSAP2, MYCBP2, HERC1, CD47, AKT2, SGRN, RAP1A, PF-4 isoform 1, PF-4 isoform 4, Rab-14, HSP70 protein 1A/1B, HSP70 protein 6, HSP70 protein 1A/1B, HSP90 isoform 2, integrin beta-3, and FKBBP1A are detected in platelets using the methods described herein.

In some embodiments, a “balance of stimulatory vs inhibitory polypeptides” can be used to predict or indicate the presence of cancer or a given stage of cancer. As an example, an increase in the amount or concentration of one or more cancer stimulatory polypeptides (e.g., above a given threshold) and/or a reduction in amount or concentration of one or more cancer inhibitory polypeptides (e.g., below a given threshold) will tip the balance towards cancer stimulation, thereby indicating that cancer is either present or progressing in a subject. Conversely, a reduction in the amount or concentration of one or more cancer stimulatory polypeptides and/or an increase in amount or concentration of one or more cancer inhibitory polypeptides will tip the balance towards cancer inhibition, thereby indicating that cancer is either not present, the cancer is not progressing in a subject, or a therapy used to treat the subject for cancer is effective.

Methods for Detection of Polypeptides Associated with Cancer Functions

Essentially any method for detecting a plurality (e.g., at least 2) of polypeptides sequestered by platelets and associated with one or more cancer functions as described herein can be used with the methods described herein. Detection of platelet-sequestered proteins can be performed using any method known in the art. While liquid-based biopsies, microfluidic devices, lateral flow assays or test strips are preferred to detect the amount of stimulators vs. inhibitors of cancer, isolation and detection of such proteins by other means is also specifically contemplated herein.

Capillary action lateral flow test strips etc: The polypeptides representative of one or more cancer functions can be detected using a testing device or kit as described herein. In one embodiment, the testing device will comprise a sample receiving zone to which a platelet or blood sample from a subject is added, and (ii) a conjugate zone comprising a plurality of labelled binding reagents, each of which specifically binds to one of the polypeptides selected from RAB1B, RAP1A, Heat shock protein 70 kDa protein 1B, Heat shock 70 kDa protein 6, Heat shock 70 kDa protein 1-like, Heat shock protein HSP 90-alpha isoform 2, MYCBP2, CD47, FKBP1A, Tribbles homolog 2, PF4, TSP-1, basic FGF, VEGF, PDGF beta, Integrin beta-3, CAMSAP2, SRGN, RANK-L, TGF-beta, IGF-1, and Synaptobrevin homolog YKT6. In another embodiment, a testing device can comprise a conjugate zone comprising a plurality of labelled binding reagents, each of which specifically binds to one of the polypeptides listed in FIG. 3 or 5. The testing device or kit can also comprise a solid support defining a liquid flow path for the sample and comprising corresponding test lines for each of the plurality (e.g., at least 2) polypeptides, each test line comprising: (a) an immobilized further binding reagent that also specifically binds to one of the plurality of polypeptides thereby immobilizing the polypeptide at the test line to produce a signal via the labelled binding reagent also specifically bound to the polypeptide; or (b) an immobilized version of one of the plurality of polypeptides or an analogue thereof able to compete with the polypeptide in the sample for specific binding to the labelled binding reagent. In some embodiments, the test lines for each polypeptide are spatially separated to permit levels of each polypeptide to be measured and discriminated from levels of the other polypeptide(s).

In particular embodiments, the boundary of the sample receiving zone can be marked for the user's convenience; for instance, using one or more symbols such as arrows. The user should dip the sample receiving zone portion of the strip into the sample up to the one or more symbols. This ensures that the sample receiving zone is sufficiently brought into contact with the sample to be tested but that the downstream components (e.g. test lines) are not.

The testing device or testing kit can further comprise at least one labelled control binding reagent that binds to a binding partner immobilized at a control line downstream of the test line(s) for the at least one or plurality of polypeptides and thus confirms that the test has completed successfully. The control line can be spatially separated from the test lines for each polypeptide. By way of illustration, in specific embodiments the binding partner immobilized at the control line comprises BSA-Biotin and the labelled control binding reagent that binds to the immobilized binding partner comprises an anti-Biotin antibody complexed with gold particles.

The testing device can further comprise an absorbent material downstream of the test (and control, where present) line(s) to absorb excess sample. In specific embodiments, the solid support comprises a chromatographic medium or a capillary flow device. The device can be provided in a test strip format in some embodiments.

In particular embodiments, a region is provided downstream of the test lines (and control line and/or absorbent material if present) which can be held by hand by the user. Thus, the user can easily manipulate the testing device without compromising the sample and subsequent testing thereof. The region can be called a “hold region” and can be made of any suitable material, such as plastic. The region can be visibly marked “hold region” or simply “hold” or similar for the user's convenience.

In some embodiments, the testing device comprises a dipstick or test strip with a plurality of testing zones, wherein each testing zone can detect a given polypeptide. When a dipstick or test strip is used, detecting a color change in the dipstick or test strip can indicate the measurement of specific analytes or polypeptides in each test zone of the panel. Each test zone can change the amount of colored light reflected from one of the components of the dipstick or can change color based on a chemical reaction. For a negative result (i.e. the presence of a given polypeptide is not detected), the strip can remain its original color, or it can change to a specific color. For a positive result (i.e. the presence of a given polypeptide is detected), the strip can change to a distinctively different color than the negative result. One example is the strip turning blue for a negative result and pink for a positive result. In some embodiments, the results are non-qualitative (color versus lack of color) but vary in degree corresponding to the level of the polypeptide present. For example, an intense color can indicate the presence of high levels of the specified polypeptide, and a muted color can indicate the presence of low levels of the polypeptide.

In some embodiments, the dipstick or other dry chemistry device can be inserted into an instrument that quantifies the reflected color for each test pad (preferably handheld) and a quantitative value can be recorded. In this method, the amount of each polypeptide present can be determined to provide further information as to the health of the subject. In other words, lower or higher levels of polypeptides, and not just their presence, can be relevant to e.g., the stage of cancer. Alternatively, a quantification device is included in the panel itself and is not a separate device.

The quantification device can include or be coupled to a computer with software that is capable of performing analysis using the data thus obtained with an analyzing mechanism. The analyzing mechanism can compute values of each of the polypeptides in the tests, perform normalization, as well as compute relationships of the test results with each other, the test results can be calculated to determine the presence and/or stage of a cancer. The analyzing mechanism can also search a database for facts relating high or low levels of the polypeptides to cancer and for appropriate treatment thereof.

The presence or amount of platelet-sequestered proteins can then be indicated to the user. The quantification device further includes an output mechanism to display the results in a meaningful way to an individual, clinical laboratory technician or health care practitioner. The display can be on a screen included on the panel and can include a printing mechanism for printing the results. Alternatively, the output mechanism can also send the results over wireless signals or wires to a PDA, smart phone, or a remote computer for print out or display. The results can be incorporated into a report on an individual's wellness or cancer risk assessment that includes, but is not limited to, the results of the tests, comparison to the values and ratios computed to normal ranges that have previously been established for normal healthy men and women of different ages, ethnicities (if relevant) and/or other relevant parameters. Such a report can also incorporate historical data for an individual subject that was obtained using the same method(s), which can be used to monitor effectiveness of treatment.

The preferred use of a given panel of polypeptides is a point-of-testing cancer risk assessment or cancer staging assessment, which can be performed in a doctor's office or other health care practitioner. The panel can also be used by individuals to monitor their health in their own home.

A desired panel of platelet-sequestered polypeptides to be tested can including the use of a single, easy-to-use, and disposable test strip that comprises multiple test zones and remove the need for individual assays for each of the various polypeptides discussed herein.

Furthermore, with the integration of all of these tests onto a single platform, it is possible to aggregate the data from all results and to compile it in a complementary way so that the data from individual test zone enhances the interpretation of other test zones on the strip. For example, the presence or amount of a single platelet-sequestered polypeptide as described herein may or may not be indicative of cancer presence or stage when viewed alone, but when combined with information detailing the amount or presence of multiple other platelet-sequestered proteins, it is now possible to identify the subject as having cancer or a particular stage of cancer.

Where the testing device is a lateral flow device, the testing device can further comprise a visual aid such as a printed document (e.g. a printed card) displaying different line intensity patterns from which the user (e.g., clinician) can interpret the results of the completed assay(s). By way of example to illustrate the concept, where the polypeptide is detected via a sandwich assay format as described herein, the lines can be graded (Grade lines 1-10) wherein Grade line 1 is the lightest colored line followed by Grade line 2 which is more intense in color and so on to Grade line 10 which is the darkest (i.e. most intensely colored) line; Grade lines 1 and 2 being calibrated at or below a pre-determined threshold level and indicating that the specific polypeptide is present but within normal parameters and therefore a cancer or function thereof is not detected whilst Grade lines 3-10, calibrated above the pre-determined threshold level, indicate that the specific polypeptide is present in abnormally high levels and therefore a cancer is detected and/or can be staged. The increasing intensities of Grade lines 3-10 enables the user, particularly when analyzing multiple samples taken overtime using the monitoring methods described herein, to understand whether the polypeptide levels are continuing to abnormally increase and therefore whether the cancer is progressing and/or current treatment is ineffective. A null grade line (Grade line 0) can also be provided for which no colored line is displayed on the visual aid indicating that the polypeptide is absent (or present at negligible levels) from the platelet or blood sample.

In some embodiments, detection of a given polypeptide or panel thereof is by way of enzymatic activity, which can be measured directly in the platelet or platelet-protein sample or using a lateral flow assay or liquid bioassay. Enzymatic activity can be measured for example by detecting processing of a substrate, which can be labelled, in the sample. For example, the assay can be a fluorogenic substrate assay. Examples of suitable assay formats include the assays set forth in International Patent Applications WO2009/024805, WO2009/063208, WO2007/128980, WO2007/096642, WO2007/096637, WO2013/156794, WO2015/059487 and WO2013/156795 (the content of each of which is hereby incorporated by reference).

In some embodiments, the determined levels of the polypeptides are compared with a corresponding threshold level. This allows an increase (or decrease) relative to the threshold to be identified. Threshold levels of a given polypeptide can be defined from population studies or be specific to the individual (i.e. personalized, or from a prior cancer risk or staging assessment). Personalized levels can be more relevant to monitoring applications, although monitoring is preferably also achieved by comparison with pre-determined threshold levels. Threshold levels can be set with reference to a training data set comprising samples defined in relation to a particular cancer status or stage. Thus, threshold levels do not need to be measured each time an assay according to the methods as performed. They can be pre-programmed into a reader device or provided for comparative purposes when performing the such methods. Because the threshold levels can vary according to the measuring technique adopted they are not stated as fixed values but can be implemented by one skilled in the art once a specific measuring technique has been selected. Where specific to the individual, the levels can reflect those in a sample taken from the subject at an earlier time point. The methods can therefore rely upon a personalized baseline level of the relevant polypeptide or polypeptides against which the threshold is calculated. Calculation can be on an on-going basis to coincide with testing. Thus, the threshold can be a rolling threshold derived from the rolling baseline. In this context, it is apparent that levels of the polypeptide or polypeptides do not have to be measured in absolute terms and can be measured in absolute or relative terms. The markers simply have to be measured in a manner which permits a comparison to be made with polypeptide levels in samples taken at different time points.

Alternatively, the threshold level for each marker can be set based on a population analysis. The threshold level can be set to maximize sensitivity and/or specificity of detection as would be readily appreciated by one skilled in the art.

In some embodiments, the threshold level of the polypeptide is set by determining the levels of the polypeptide in samples taken from the subject at earlier time points. In its simplest form, the methods can rely upon a simple comparison between the test sample and the level of the polypeptide in the previously taken sample (i.e. a single earlier time point). However, the earlier time points can comprise at least two, and possibly 3, 4, 5, 6, 7, 8, 9, 10 etc, earlier measurements immediately preceding the determination of the level of the polypeptide in the current sample.

Isolation and Detection of proteins sequestered by platelets: If desired, proteins can be isolated from platelets as described in “Current Protocols in Immunology by F. M. Ausubel, R. Brent, R. E. Kingston, D. D. Moore, J. G. Seidman, K. Struhl and V. B. Chanda (Editors), John Wiley & Sons, 2004.”, incorporated herein by reference. In one example, described in WO 02/077176, also incorporated herein by reference, the procedure generally involves the extraction of proteins in one solubilizing step. The results of this procedure are intact proteins, substantially free of cross-contamination. The isolated proteins maintain activity, allowing analysis through any number of assays.

The buffers for the protein isolation step can include one or more of buffer components, salt (s), detergents, protease inhibitors, and phosphatase inhibitors. In particular, one effective buffer for extracting proteins to be analyzed by immunohistochemistry includes the buffer Tris-HCl, NaCl, the detergents Nonidet (g) P-40, EDTA, and sodium pyrophosphate, the protease inhibitors aprotinin and leupeptin, and the phosphatase inhibitors sodium deoxycholate, sodium orthovanadate, and 4-2 aminoethylbenzenesulfonylfluroride (AEBSF). Another salt that could be used is LiCl, while glycerol is a suitable emulsifying agent that can be added to the fraction buffer. Additional optional protease inhibitors include soybean trypsin inhibitor and pepstatin. Other suitable phosphatase inhibitors include phenylmethylsufonyl fluoride, sodium molybdate, sodium fluoride, and betaglycerol phosphate.

For 2-D gel analysis, simple lysis with a I % SDS solution is effective, while ultimate analysis using the SELDI® process requires Triton-X-100, a detergent (Sigma, St. Louis, Mo.), MEGA109 (ICN, Aurora, Ohio), and octyl B-glucopyranoside (ESA, Chelmsford, Mass.) in a standard PBS base. Another buffer which was used prior to 2-D gel analysis was 7M urea, 2M thiourea, CHAPS, MEGA 10, octyl B-glucopyranoside, Tris, DTT, tributyl phosphine, and Pharmalytes.

Once the proteins have been solubilized, a number of different immunological or biochemical analyses can be used to characterize the isolated proteins. Methods for analysis by ELISA and Western blot are known to those of skill in the art and are further described in “Current Protocols in Molecular Biology by F. M. Ausubel, R. Brent, R. E. Kingston, D. D. Moore, J. G. Seidman, K. Struhl and V. B. Chanda (Editors), John Wiley & Sons, 2004”, incorporated herein by reference. Methods of performing mass spectrometry are known to those of skill in the art and are further described in Methods of Enzymology, Vol. 193: “Mass Spectrometry” (J. A. McCloskey, editor), 1990, Academic Press, New York.

The proteins sequestered by platelets, as described herein, can be used to predict, indicate, diagnose, or monitor progression or regression of cancer and can be measured using any process known to those of skill in the art including, but not limited to, enzyme linked immunosorbent assay (ELISA), fluorescence polarization immunoassay (FPIA) and homogeneous immunoassays, point of care tests using conventional lateral flow immunochromatography (LFA), quantitative point of care tests using determination of chemiluminescence, fluorescence, and magnetic particles, as well as latex agglutination, biosensors, gel electrophoresis, mass spectrometry (MS), gas chromatograph-mass spectrometry (GC-MS), nanotechnology based methods, proximity extension assays (e.g., the use of DNA oligonucleotides linked to antibodies against a target molecule that can be quantified with real-time polymerase chain reaction), slow-offrate-modified-aptamer reagent (SOMAmer) assays, nanoscale needles etc. Such technologies can include immunofluorescent assays, enzyme immunoassays, radioimmunoassays, chemiluminescent assays, sandwich-format assays, techniques using microfluidic or MEMS technologies, re-engineering technologies (e.g. instruments utilizing sensors for polypeptides used for telemedicine purposes), epitope-based technologies, other fluorescence technologies, microarrays, lab-on-a-chip, and rapid point-of-care screening technologies.

Exemplary proximity extension assays are available commercially from Olink (Uppsalla, Sweden), while exemplary slow-offrate-modified-aptamer reagent (SOMAmer) assays include SOMAscan™ assays available commercially from SOMAlogic (Boulder, CO). Exemplary assays using nanoscale needles that function as label free biosensors, functionalized with capture antibodies that change color once bound to its target (which is then quantified) is available commercially from companies such as NanoMosaic (Woburn, MA).

Other detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample can be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

“Protein biochip” refers to a biochip adapted for the capture of polypeptides. Many protein biochips are described in the art. These include, for example, protein biochips produced by Ciphergen Biosystems, Inc. (Fremont, Calif), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828.

In another embodiment, the polypeptides described herein are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these. In one embodiment, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

In another embodiment, the mass spectrometric technique for use in detecting the polypeptides described herein is “Surface Enhanced Laser Desorption and Ionization” or “SELDI.” This refers to a method of desorption/ionization gas phase ion spectrometry (e.g., mass spectrometry) in which the polypeptides are each captured on the surface of a SELDI mass spectrometry probe. There are several versions of SELDI known to those of skill in the art and contemplated for use herein, including “affinity capture mass spectrometry” or surface-enhanced affinity capture (SEAC), surface-enhanced neat desorption (SEND), surface-enhanced photolabile attachment and release (SEPAR).

In another embodiment, a mass-spectrometry method can be used to capture a polypeptide on an appropriate chromatographic resin. For example, one could capture the polypeptides on a cation exchange resin, such as CM Ceramic HyperD F resin, wash the resin, elute the polypeptides and detect by MALDI. Alternatively, this method can be preceded by fractionating the sample on an anion exchange resin before application to the cation exchange resin. In another alternative, one could fractionate on an anion exchange resin and detect by MALDI directly. In yet another method, one could capture the polypeptides on an immuno-chromatographic resin that comprises antibodies that bind the polypeptides, wash the resin to remove unbound material, elute the polypeptides from the resin and detect the eluted polypeptides by MALDI or by SELDI. These methods are known to those of skill in the art and are not described in detail herein.

In another embodiment, the polypeptides described herein can be detected and/or measured by immunoassay, where specific capture reagents, such as antibodies or binding fragments thereof, to bind each of the polypeptides in the panel. Antibodies can be produced by methods well known in the art, e.g., by separately immunizing animals with each of the polypeptides. Polypeptides can be isolated from samples based on their binding characteristics. Alternatively, if the amino acid sequence of a polypeptide polypeptide is known, the polypeptide can be synthesized and used to generate antibodies by methods well known in the art.

Also specifically contemplated herein are traditional immunoassays for detecting or measuring the level of a polypeptide in the panel including, but not limited to, sandwich immunoassays including ELISA or fluorescence-based immunoassays, as well as other enzyme immunoassays. In the SELDI-based immunoassay, an antibody or other binding reagent for the polypeptide is attached to the surface of an MS probe, such as a pre-activated ProteinChip array. The polypeptide can then be specifically captured on the biochip and detected by mass spectrometry.

Detection of a given polypeptide can require the use of a label or detectable moiety. Such detectable moieties can be isotopic labels; magnetic, electrical or thermal labels; colored or luminescent dye; and enzymes, all of which enable detection of the polypeptide(s). In various embodiments, a secondary detectable label is used. A secondary label is one that is indirectly detected including, but not limited to, one of a binding partner pair; chemically modifiable moieties; nuclease inhibitors; enzymes such as horseradish peroxidase, alkaline phosphatases, luciferases etc. In certain sandwich formats, an enzyme can serve as the secondary label, bound to the soluble capture ligand. In various embodiments, the system relies on detecting the precipitation of a reaction product or on a change on the properties of the label, for example the color for detection. A detection system for colorimetric methods can include a spectrophotometer, a colorimeter, or other device that measures absorbance or transmission of light on one or more wavelengths.

As described herein, assessment of results can be qualitative or quantitative depending upon the specific method of detection employed.

While the methods described herein are exemplified using analysis at the protein level, it is specifically contemplated herein that the step of determining expression of each member in a panel can be detected at the mRNA level can also be used with the methods described herein.

### Reference Levels

The results of a polypeptide or polypeptide panel from a given biological sample (e.g., a platelet sample or a sample of proteins isolated from platelets) can be compared to those of a control biological sample tested using substantially the same methods or to a visual reference card for dipstick or lateral flow style assays. Comparing the expression level or amount of each member in the panel of polypeptides in a biological sample from a subject suspected of having cancer with the expression level or amount of each member in the panel of polypeptides in a control biological sample (e.g., from a subject or population of subjects lacking detectable cancer) can permit diagnosis or staging of cancer in the subject. Control biological samples can also be a reference sample taken from the subject at an earlier time point (e.g., during initial diagnosis of a given cancer or as a baseline prior to initiation of cancer treatment) to permit monitoring of disease progress in the subject. For example, the control biological sample can be a sample taken from the subject one month, two months, three months, six months, or one year prior to the sample to be tested. In other embodiments, the control biological sample or reference sample is obtained from the subject during, or following the administration of a given anti-cancer therapy. In other aspects, a reference sample can be a sample from a patient or a population of patients having no detectable cancer (i.e., a negative control). Alternatively, a reference sample can be a sample from a patient or population of patients with a known stage of cancer (e.g., prostate cancer), for example, mild, moderate or advanced cancer (e.g., prostate cancer) or specific stages such as T2a or T3a prostate cancer. It should be understood that there can be more than one reference in a given assay. Such controls can serve as a positive control to ensure that the assay is working and/or permit “staging” of a given cancer. Thus, in other embodiments, a reference is from a subject known to have cancer. In one embodiment, the cancer is the same cancer that is being monitored using the methods described herein. In another embodiment, the reference is from a different cancer from that being tested using the methods described herein. In some embodiments, the reference is from a subject having a secondary cancer or a plurality of different cancers. In other embodiments, one or more controls can comprise a known concentration (or range of concentrations) of each of the polypeptides in the panel in order to quantitatively detect the level of each polypeptide in the subject being tested. In certain embodiments, a positive or negative control sample is a sample that is obtained or derived from a corresponding tissue or biological fluid or tumor as the sample to be analyzed in accordance with the methods as described herein. This sample will typically be from the same patient at the same or different time points.

In certain embodiments the level of one or more of the polypeptides described herein can be compared to a reference value or the level the polypeptide in a control or reference sample in order to assess the “risk” of a subject for developing cancer. In other embodiments, the level of polypeptides in the panel described herein can be compared to a reference value in order to determine the prognosis of a subject (i.e., potential for survival).

In another embodiment the reference levels for one or more polypeptides are established based on polypeptide levels in a sample taken from an individual at an earlier point in time, for example, prior to onset of treatment with a therapeutic agent. Such methods permit one of skill in the art to monitor the efficacy of a given therapeutic. The individual is determined to be responding to treatment for cancer if the relative amounts of the polypeptides in the biological sample have altered favorably from the polypeptide levels in a biological sample taken at an earlier first time point from the same individual; i.e. trend towards normal polypeptide levels. Similarly, the disease state of the individual may be progressing if the polypeptide levels in a biological sample are changing relative to the levels in the individual taken at an earlier time point or in reference to the control levels. In some embodiments, where a subject is determined to be worsening despite treatment with a given anti-cancer agent, the methods provided herein permit one of skill in the art to shift the subject to treatment with a more aggressive chemotherapeutic.

### Algorithms and Normalization

For those embodiments which rely upon measuring the levels of a plurality of polypeptides, the final detection or staging of cancer can require that the measured levels are integrated, ideally to provide a simply binary result that is readily interpreted. A suitable algorithm can be employed in order to interpret the data from the levels of the plurality of polypeptides (e.g., at least 2, 3, 4, 5, 6, 7, 8, 9, 10, 11, 12, 13, 14, 15, or 16 polypeptides in Table 5 or FIG. 3 or 5) and apply it in order to detect or stage cancer (e.g., prostate cancer). In some embodiments, the polypeptides levels can be inter-dependent and thus the algorithm is based on this predicted relationship. In certain embodiments, the determined levels of the plurality of polypeptides are analyzed in a pre-determined sequence to monitor the subject. This may give rise to a decision tree to detect or stage a cancer. The levels of the first of the multiple polypeptides can influence the subsequent thresholds required for the other polypeptides in order to detect or stage a cancer, as would readily be appreciated by one skilled in the art. The output of the methods can also guide future sampling and treatment of the subject.

In some embodiments, the determined levels of the plurality of polypeptides (e.g., at least 1 and up to and including all 16 polypeptides) are weighted. Weighting is a well-known method of applying a degree of relative significance to each polypeptide in the plurality of polypeptides. The algorithm can be a threshold based algorithm as discussed herein.

The levels of the measured polypeptides can be combined using logistic regression, decision tree analysis, neural networks and/or machine learning. Logistic regression analysis involves formulating a statistical model which adds different markers together in a weighted fashion. Similar to linear regression which can be resolved using the equation y=mx+c, logistic regression allows for the addition of multiple polypeptides and only allows for a binary outcome so the y is replaced with the logit (defined as the In(odds) of being in the positive outcome group). Mathematically, the logistic regression equation is logit=(βnXn)+c. This uses quantitative data of all polypeptides in a weighted fashion in the calculation.

In decision tree analysis, an individual is assessed for one polypeptide at a time until they reach a terminal node which classifies the patient into the positive or negative outcome group. This uses cut-off values for each individual polypeptide and does not necessarily use all polypeptides in the algorithm, depending upon at which point they are categorized. This type of analysis is therefore suitable for embodiments of the invention in which the levels of more than one polypeptide are determined.

Use of neural network displays some similarity to logistic regression in that each node is a summation of the input (marker) multiplied by a weighting (beta coefficient). However, summation is performed a number of times; there are a number of nodes and the input to these nodes can be nodes themselves rather than the measured levels. The nodes are first entered at random with random weights, the difference between the expected output and the observed output is then calculated. If it is not 0 (which is likely to be the case) the weightings are altered in the preceding layer and then in the layer before that until the input variables are reached. The outputs are recalculated and the differences are calculated again, and the model weighting readjusted. This can continue indefinitely until the difference in expected and observed outputs is minimal.

### Anti-Cancer Therapies

Any anti-cancer therapy which is useful, has been used, is currently being used, or can be used for the prevention, treatment and/or management of cancer can be used to prevent, treat, and/or manage cancer in accordance with the methods and assays described herein. Exemplary anti-cancer agents include, but are not limited to, peptides, polypeptides, fusion proteins, nucleic acid molecules, small molecules, mimetic agents, synthetic drugs, inorganic molecules, and organic molecules. Non-limiting examples of cancer therapies include chemotherapies, radiation therapies, hormonal therapies, anti-angiogenesis therapies, targeted therapies, and/or biological therapies including immunotherapies and surgery. In certain embodiments, a therapeutically effective regimen comprises the administration of a combination of at least two therapies or at least two agents.

Examples of anti-cancer therapies include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracyclin; anthramycin; asparaginase; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandomate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine (Ara-C); dacarbazine; dactinomycin; daunorubicin hydrochloride; decitabine (Dacogen); demethylation agents, dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; histone deacetylase inhibitors (HDAC-Is) hydroxyurea; idarubicin hydrochloride; ifosfamide; ilmofosine; imatinib mesylate (Gleevec, Glivec); interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; lenalidomide (Revlimid); letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper, mitotane; mitoxantrone hydrochloride; mycophenolic acid; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate: triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Additional exemplary anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TIC antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin Ill derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor, cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-Ntriacetate; lanrcotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor, mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor, multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; oracin; oral cytokine inducer, ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; gamma secretase inhibitors, single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; S-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; anti-integrin antibodies (e.g., anti-integrin αvβ3 antibodies); vorozole; zanoterone; zeniplatin; zilascorb; and zinostatin stimalamer.

In some embodiments, an anti-cancer agent is administered in combination with a steroid or other therapeutic agent.

### Pharmaceutical Compositions, Administration and Efficacy

Pharmaceutical or therapeutic compositions comprising a therapeutic agent for the treatment of cancer can contain a physiologically tolerable carrier, wherein the therapeutic agent is dissolved or dispersed therein as an active ingredient(s). In a preferred embodiment, the pharmaceutical composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological or pharmaceutical composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition comprising a therapeutic agent for treatment of cancer can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of cancer or a symptom thereof will depend on the nature of the disorder or condition, and can be determined by standard clinical techniques.

A pharmaceutical composition as described herein can be formulated for parenteral administration, e.g., by bolus injection or continuous infusion. Formulations for injection can be presented in unit dosage form, e.g., in ampoules or in multidose containers with, optionally, an added preservative. The compositions can be suspensions, solutions, or emulsions in oily or aqueous vehicles, and can contain formulatory agents such as suspending, stabilizing, and/or dispersing agents.

Pharmaceutical compositions for parenteral administration include aqueous solutions of the active preparation in water-soluble form. Additionally, suspensions of the active ingredients can be prepared as appropriate oily or water-based injection suspensions.

Suitable lipophilic solvents or vehicles include fatty oils such as sesame oil, or synthetic fatty acid esters such as ethyl oleate, triglycerides, or liposomes. Aqueous injection suspensions can contain substances that increase the viscosity of the suspension, such as sodium carboxymethyl cellulose, sorbitol, or dextran.

Optionally, the suspension can also contain suitable stabilizers or agents that increase the solubility of the active ingredients, to allow for the preparation of highly concentrated solutions. Alternatively, the active ingredient can be in powder form for constitution with a suitable vehicle, e.g., a sterile, pyrogen-free, water-based solution, before use.

In some embodiments, a therapeutic agent can be delivered in an immediate release form. In other embodiments, the therapeutic agent can be delivered in a controlled-release system or sustained-release system. Controlled- or sustained-release pharmaceutical compositions can have a common goal of improving drug therapy over the results achieved by their non-controlled or non-sustained-release counterparts. Advantages of controlled- or sustained-release compositions include extended activity of the therapeutic agents, reduced dosage frequency, and increased compliance. In addition, controlled- or sustained-release compositions can favorably affect the time of onset of action or other characteristics, such as blood levels of the therapeutic agent, and can thus reduce the occurrence of adverse side effects. Controlled- or sustained-release of an active ingredient can be stimulated by various conditions, including but not limited to, changes in pH, changes in temperature, concentration or availability of enzymes, concentration or availability of water, or other physiological conditions or compounds.

In one embodiment, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)). In yet another embodiment, a controlled- or sustained-release system can be placed in proximity of a target of infection, e.g., bone marrow, thus requiring only a fraction of the systemic dose.

When in tablet or pill form, a pharmaceutical composition as described herein can be coated (e.g., enterically coated) to delay disintegration and absorption in the gastrointestinal tract, thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

The pharmaceutical composition as described herein can also be formulated in rectal compositions such as suppositories or retention enemas, using, for example, conventional suppository bases such as cocoa butter or other glycerides.

The appropriate dosage range for a given therapeutic agent depends upon the potency, and includes amounts large enough to produce the desired effect, e.g., reduction in at least one symptom of cancer. The dosage of the therapeutic agent should not be so large as to cause unacceptable or life-threatening adverse side effects or should be used under close supervision by a medical professional. Generally, the dosage will vary with the type of anti-cancer agent, and with the age, condition, and sex of the patient. The dosage can be determined by one of skill in the art and can also be adjusted by the individual physician in the event of any complication.

Typically, the dosage of a given therapeutic can range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 g/mL and 30 g/mL.

Currently available therapies, including experimental therapies, for cancer or a symptom thereof and their dosages, routes of administration and recommended usage are known in the art and/or have been described in such literature as the Physician's Desk Reference (60th ed., 2017). With respect to experimental therapies, an appropriate dosage can be estimated based on dose-response modeling in animal models or in silico modeling of drug effects.

Administration of the doses recited above or as employed by a skilled clinician can be repeated for a limited and defined period of time. In some embodiments, the doses are given once a day, or multiple times a day, for example, but not limited to three times a day. Typically, the dosage regimen is informed by the half-life of the agent as well as the minimum therapeutic concentration of the agent in blood, serum or localized in a given biological tissue. In a preferred embodiment, the doses recited above are administered daily for several weeks or months. The duration of treatment depends upon the subject's clinical progress and continued responsiveness to therapy. Continuous, relatively low maintenance doses are contemplated after an initial higher therapeutic dose.

A therapeutically effective amount is an amount of an agent that is sufficient to produce a statistically significant, measurable change of a given symptom of cancer (see “Efficacy Measurement” below). Such effective amounts can be gauged in clinical trials as well as animal studies for a given agent. For example, reduction of a given symptom of cancer can be indicative of adequate therapeutic efficacy of an agent(s).

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired.

Therapeutic compositions containing at least one therapeutic agent can be conventionally administered in a unit dose. The term “unit dose” when used in reference to a therapeutic composition refers to physically discrete units suitable as unitary dosage for the subject, each unit containing a predetermined quantity of a therapeutic agent calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

In some embodiments, a combination of anti-cancer therapeutic agents is used in the treatment of cancer in a subject diagnosed as described herein.

In some embodiments, a therapeutically effective agent is administered to a subject concurrently with a combination therapy. As used herein, the term “concurrently” is not limited to the administration of the two or more agents at exactly the same time, but rather, it is meant that they are administered to a subject in a sequence and within a time interval such that they can act together (e.g., synergistically to provide an increased benefit than if they were administered otherwise). For example, the combination of therapeutics can be administered at the same time or sequentially in any order at different points in time; however, if not administered at the same time, they should be administered sufficiently close in time so as to provide the desired therapeutic effect, preferably in a synergistic fashion. The agents can be administered separately, in any appropriate form and by any suitable route. When each of the therapeutic agents in a combination are not administered in the same pharmaceutical composition, it is understood that they can be administered in any order to a subject in need thereof. For example, the first therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second therapeutic agent, to a subject in need thereof (or vice versa). In other embodiments, the delivery of either therapeutic agent ends before the delivery of the other agent/treatment begins. In some embodiments of either case, the treatment is more effective because of combined administration. For example, the therapeutic agents used in combination are more effective than would be seen with either agent alone. In some embodiments, delivery is such that the reduction in a symptom, or other parameter related to the disorder is greater than what would be observed with either therapeutic agent alone. The effect of such a combination can be partially additive, wholly additive, or greater than additive. The agent and/or other therapeutic agents, procedures or modalities can be administered during periods of active disease, or during a period of persistence or less active disease.

When administered in combination, one or more of the therapeutic agents can be administered in an amount or dose that is higher, lower or the same as the amount or dosage of the given agent used individually, e.g., as a monotherapy. In certain embodiments, the administered amount or dosage of a first therapeutic agent when administered in combination with a second therapeutic agent is lower (e.g., at least 20%, at least 30%, at least 40%, or at least 50%) than the amount or dosage of the first agent when used individually. In other embodiments, the amount or dosage of a first therapeutic agent, when administered in combination with a second therapeutic agent, results in a desired effect (e.g., improved cognitive functioning) is lower (e.g., at least 20%, at least 30%, at least 40%, or at least 50% lower) than the amount or dosage of the first (or second) agent required to achieve the same therapeutic effect when administered alone.

The efficacy of a given treatment for cancer can be determined by the skilled clinician. However, a treatment is considered “effective treatment,” as the term is used herein, if any one or all of the signs or symptoms of cancer is/are altered in a beneficial manner, or other clinically accepted symptoms or markers of disease are improved, or ameliorated, e.g., by at least 10% following treatment with a therapeutic agent for cancer. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the cancer; or (2) relieving the disease, e.g., causing regression of symptoms; and (3) preventing or reducing the likelihood of the development of the disease, or preventing secondary diseases/disorders associated with the infection (e.g., anemia).

An effective amount for the treatment of a disease means that amount which, when administered to a mammal in need thereof, is sufficient to result in effective treatment as that term is defined herein, for that disease. Efficacy of an agent can be determined by assessing physical indicators of the disease, such as e.g., anemia, white blood cell levels or identity, pain, fatigue, fever, etc.

The treatment according to the methods provided herein can reduce or eliminate one or more symptoms associated with cancer such as fatigue, pain, tumor size, tumor growth, etc. In one embodiment, the cancer is prostate cancer and the one or more symptoms associated with prostate cancer include trouble urinating, increased frequency of urination, pelvic pain or discomfort, decreased force of urination, difficulty starting or stopping urine stream, blood in semen, and bone pain.

### Devices and Kits

Provided herein are kits that include quantification of individual platelet-associated polypeptides or a subset of platelet-sequestered proteins using, e.g. an antibody test from whole blood targeting proteins as described herein sequestered by platelets. The kits can optionally include an agent or device for quantification of the platelet proteins. For example, the kits can optionally include agents or devices for, collection of whole blood, lysing platelets, such as, e.g., (0.01% Triton X), and quantification such as, e.g., (by flow cytometry or chemical assay). In other examples, the kits can include one or more sterile applicators, such as syringes or needles. In addition, the kits can optionally include other agents, for example, anesthetics or antibiotics. The kit can also include a package insert that instructs a user of the kit, such as, e.g., (a physician, laboratory technician) to perform the methods disclosed herein.

In some embodiments, the testing device comprises disposable single use devices to which the sample is applied. Typically, such a testing device can comprise a sample receiving zone to which the sample is added. These devices typically also incorporate a solid support which defines a liquid/capillary flow path for the sample once applied to the sample receiving zone. The sample receiving zone can be an integral part of the solid support. The solid support can comprise a chromatographic medium, such as a membrane material in some embodiments (e.g. nitrocellulose). A sample applied to the sample application zone will typically rehydrate the necessary reagents to detect the marker. The reagents include binding reagents which specifically interact with the polypeptides or a substrate for effector molecules where activity is measured. Further reagents immobilized further along the flow path bind to the complex of polypeptide and binding reagent. The binding reagent is labelled to provide a signal at the site of immobilization of the complex of polypeptide and binding reagent (through binding to the further reagent). Suitable labels include fluorescent labels, magnetic labels, latex or gold as would be readily understood by one skilled in the art.

When enzymatic activity is being assayed the binding reagent and/or further binding reagent can bind with a substrate only after it has been modified by the enzymatic activity, or can only bind if the substrate has not been modified by the enzymatic activity. Examples of enzymatic activity assays include those set forth in International Patent Applications WO2009/024805, WO2009/063208, WO2007/128980, WO2007/096642, WO2007/096637, WO2013/156794, WO2015/059487 and WO2013/156795 (the content of each of which is hereby incorporated by reference).

The binding reagent and further reagent are typically antibodies. Thus, in specific embodiments, the one or more testing devices, testing kits or testing compositions of matter can comprise a lateral flow test strip. In some embodiments, a single lateral flow test strip is employed to permit detection of all polypeptides that are to be determined in the test sample. In other embodiments, a separate lateral flow test strip is provided for each polypeptide that is determined.

The devices, kits or compositions of matter can also include a control zone to confirm sample has passed through the device satisfactorily. In the event this is not the case the system or test kit or reader of the testing device can indicate an invalid result to the user, for example via the display. The devices, kits or compositions of matter can act as competitive or sandwich assays. ELISA (enzyme linked immunosorbent assay) is an example of a suitable assay format that can be incorporated in the testing devices used with the methods described herein. Again, typically all reagents to detect the levels of the plurality of polypeptides are pre-loaded onto the testing device, kit or composition of matter such that they can interact with the sample once added to the device (for example via the sample receiving zone). This minimizes intervention and thus error caused by the subject. Thus, effectively, the device can only require the user to apply the sample and subsequently observe the output of the assay.

The systems, test kits, testing devices and testing compositions of matter can incorporate a suitable reader to provide a quantitative output (in conjunction with the processor and storage medium); this output can be an absolute or a relative output. Suitable readers can incorporate an illuminator to expose the device to a specific wavelength or wavelengths of light and a suitable detector for the reflected or emitted light. The systems, test kits, testing devices and testing compositions of matter can also incorporate a suitable processor and computer application to output the results based upon the detected signal. Thus, the processor running the computer application will be in operable connection with the reader. By “operable connection” is meant a functional connection that permits the exchange of a signal or information between the elements.

The one or more testing devices, kits or compositions of matter can comprise an enzyme detection device. These devices can be particularly useful for investigating enzymatic activity.

The system or test kit can incorporate the appropriate number of testing devices to permit each polypeptide to be determined. This is particularly the case where the polypeptides are detecting using different platforms. Thus, in some embodiments, the one or more testing devices comprise one or more lateral flow activity assays, ELISAs, fluorogenic substrate assays etc. In some embodiments, the one or more testing devices comprise one or more lateral flow activity assays, ELISAs or competition assays. In some embodiments, the one or more testing devices comprise one or more lateral flow assays and ELISAs.

### Computer Systems

Provided herein are testing devices and kits for performing the methods described herein. Also provided herein are computer applications for use with the systems and test kits. The computer applications can also be used in the testing devices or testing kits described herein, for example, by incorporation of a reader. Thus, in certain embodiments, the computer-implemented method, system, and computer program product can be embodied in a computer application, for example, that operates and executes on a processor, such as in the context of a computing machine.

As used herein, the processor can be comprised within any computer, server, embedded system, or computing system. The computer can include various internal or attached components such as a system bus, system memory, storage media, input/output interface, and a network interface for communicating with a network, for example.

The computer can be implemented as a conventional computer system, an embedded controller, a laptop, a server, a customized machine, any other hardware platform, such as a laboratory computer or device, for example, or any combination thereof. The computing machine can be a distributed system configured to function using multiple computing machines interconnected via a data network or bus system, for example.

The processor can be configured to execute code or instructions to perform the operations and functionality described herein, manage request flow and address mappings, and to perform calculations and generate commands. The processor can be configured to monitor and control the operation of the components in the computing machine. The processor can be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor can be a single processing unit, multiple processing units, a single processing core, multiple processing cores, special purpose processing cores, co-processors, or any combination thereof. According to certain example embodiments, the processor, along with other components of the computing machine, can be a virtualized computing machine executing within one or more other computing machines.

The storage medium can be selected from a flash memory, other non-volatile memory device, a solid-state drive (“SSD”), any magnetic storage device, any optical storage device, any electrical storage device, any semiconductor storage device, any physical-based storage device, any other data storage device, or any combination or multiplicity thereof. The storage media can store one or more operating systems, application programs and program modules such as module, data, or any other information. The storage media can be part of, or connected to, the computing machine. The storage media can also be part of one or more other computing machines that are in communication with the computing machine, such as servers, database servers, cloud storage, network attached storage, and so forth.

The storage media can therefore represent examples of machine or computer readable media on which instructions or code can be stored for execution by the processor. Machine or computer readable media can generally refer to any medium or media used to provide instructions to the processor. Such machine or computer readable media associated with the module can comprise a computer software product.

The input/output (“I/O”) interface can be configured to couple to one or more external devices, to receive data from the one or more external devices, and to send data to the one or more external devices. Such external devices along with the various internal devices can also be known as peripheral devices. The I/O interface can include both electrical and physical connections for operably coupling the various peripheral devices to the computing machine or the processor. The I/O interface can be configured to communicate data, addresses, and control signals between the peripheral devices, the computing machine, or the processor. The I/O interface can be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface can be configured to implement only one interface or bus technology.

Alternatively, the I/O interface can be configured to implement multiple interfaces or bus technologies. The I/O interface can be configured as part of, all of, or to operate in conjunction with, the system bus. The I/O interface can include one or more buffers for buffering transmissions between one or more external devices, internal devices, the computing machine, or the processor.

The I/O interface can couple the computing machine to various input devices including mice, touch-screens, scanners, electronic digitizers, sensors, receivers, touchpads, trackballs, cameras, microphones, keyboards, any other pointing devices, or any combinations thereof. The I/O interface can couple the computing machine to various output devices including video displays, speakers, printers, projectors, tactile feedback devices, automation control, robotic components, actuators, motors, fans, solenoids, valves, pumps, transmitters, signal emitters, lights, and so forth.

The computing machine can operate in a networked environment using logical connections through the network interface to one or more other systems or computing machines across the network. The network can include wide area networks (WAN), local area networks (LAN), intranets, the Internet, wireless access networks, wired networks, mobile networks, telephone networks, optical networks, or combinations thereof. The network can be packet switched, circuit switched, of any topology, and can use any communication protocol. Communication links within the network can involve various digital or an analog communication media such as fiber optic cables, free-space optics, waveguides, electrical conductors, wireless links, antennas, radio-frequency communications, and so forth.

The processor can be connected to the other elements of the computing machine or the various peripherals discussed herein through the system bus. It should be appreciated that the system bus can be within the processor, outside the processor, or both. According to some embodiments, any of the processor, the other elements of the computing machine, or the various peripherals discussed herein can be integrated into a single device such as a system on chip (“SOC”), system on package (“SOP”), or ASIC device.

Embodiments can comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement one or more of the disclosed embodiments described herein. Therefore, disclosure of a particular set of program code instructions is not considered necessary for an adequate understanding of how to make and use the embodiments. Further, those skilled in the art will appreciate that one or more aspects of embodiments described herein can be performed by hardware, software, or a combination thereof, as can be embodied in one or more computing systems. Moreover, any reference to an act being performed by a computer should not be construed as being performed by a single computer as more than one computer can perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

The methods, systems, test kits, testing devices, testing kits and testing compositions of matter can incorporate means for Automatic Identification and Data Capture (AIDC), such as a Radio-frequency identification tag or card (RIF)

In specific embodiments, the systems, test kits, testing devices or testing kits further comprise a display for the output from the processor. This is intended to give a simple visual and/or audible read-out of the assays performed on the sample. The display can be operably connected to the processor running the computer application. The output or read-out can be an instruction to the subject in some embodiments. The output can be color-coded or numerical to reflect the various possible outcomes of monitoring as discussed herein. A combination of both types of quantitative and qualitative information can be presented in some embodiments. Thus, the display can present both quantitative and qualitative read-outs in some embodiments. Probability values related to the predictive and identification outcomes can also represent an output in some embodiments. The display is typically an integral part of the reader device.

In some embodiments, the present technology may be defined in any of the following numbered paragraphs:
1. A method of diagnosing cancer in a subject, the method comprising:
   (a) measuring, in a sample of platelets from the subject, the levels of a set of polypeptides comprising subsets of polypeptides that are representative of functions in cancer development including each of malignant cell transformation, invasion, angiogenesis and metastasis;
   (b) comparing the levels of the set of polypeptides measured in step (a) with the levels of the same set of polypeptides in a reference preparation of platelets, plasma, circulating cells or to a reference value or range thereof for each polypeptide;
   wherein a difference in the level of polypeptides representative of one or more of the functions relative to the reference is indicative of cancer status in the subject.
2. The method of item 1, wherein the set of polypeptides comprises a plurality of polypeptides associated with each function.
3. The method of item 1 or item 2, wherein the polypeptides representative of functions in cancer development include cancer stimulatory and/or cancer inhibitory polypeptides, and wherein cancer progression is indicated when the balance of cancer stimulatory vs cancer inhibitory polypeptides is disrupted such that cancer stimulatory polypeptides are increased and/or cancer inhibitory polypeptides are decreased relative to the reference.
4. The method of any one of items 1-3, wherein an increase in one or more cancer stimulatory polypeptides or a decrease in one or more cancer inhibitory polypeptides is indicative of cancer progression.
5. The method of any one of items 1-3, wherein a decrease in one or more cancer stimulatory polypeptides or an increase in one or more cancer inhibitory polypeptides is indicative of cancer regression or therapy responsiveness.
6. The method of any one of items 1-5, wherein the method diagnoses prostate cancer, and the polypeptides representative of function in malignant cell transformation include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.
7. The method of any one of items 1-6, wherein the method diagnoses prostate cancer, and the polypeptides representative of functions in malignant cell transformation include two or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.
8. The method of any one of items 1-7, wherein the method diagnoses prostate cancer, and the polypeptides representative of functions in malignant cell transformation include each of RAB1B, RAP1A, Heat shock protein 70 kDa protein 1B, Heat shock 70 kDa protein 6, Heat shock 70 kDa protein 1-like, and Heat shock protein HSP 90-alpha isoform 2.
9. The method of any one of items 1-8, wherein the method diagnoses prostate cancer, and the polypeptides representative of function in cancer cell invasion include one or more of MYCBP2, CD47, FKBP1A, and Tribbles homolog 2.
10. The method of any one of items 1-9, wherein the method diagnoses prostate cancer, and the polypeptides representative of function in cancer cell invasion include two or more of MYCBP2, CD47, FKBP1A, and Tribbles homolog 2.
11. The method of any one of items 1-10, wherein the method diagnoses prostate cancer, and the polypeptides representative of function in angiogenesis include one or more of PF4, TSP-1, basic FGF, VEGF, PDGF beta, and Integrin beta-3.
12. The method of any one of items 1-11, wherein the method diagnoses prostate cancer, and the polypeptides representative of function in angiogenesis include two or more of PF4, TSP-1, basic FGF, VEGF, PDGF beta, and Integrin beta-3.
13. The method of any one of items 1-12, wherein the cancer is prostate cancer, and the polypeptides representative of function in angiogenesis include each of PF4, TSP-1, and Integrin beta-3 and optionally basic FGF, VEGF, and/or PDGF beta.
14. The method of any one of items 1-13, wherein the method diagnoses prostate cancer, and the polypeptides representative of function in metastasis include one or more of CAMSAP2, SRGN, RANK-L, TGF-beta, IGF-1, and Synaptobrevin homolog YKT6.
15. The method of any one of items 1-14, wherein the method diagnoses prostate cancer, and the polypeptides representative of function in metastasis include two or more of CAMSAP2, SRGN, RANK-L, TGF-beta, IGF-1, and Synaptobrevin homolog YKT6.
16. The method of any one of items 1-15, wherein the method diagnoses prostate cancer, and the polypeptides representative of function in metastasis include each of CAMSAP2, SRGN, and Synaptobrevin homolog YKT6 and optionally one or more of RANK-L, TGF-beta, or IGF-1.
17. The method of any one of items 1-16, wherein the aggressiveness or stage of a cancer is indicated by which of the recited functional classes of polypeptides fall within a reference range for subjects having a particular stage of cancer.
18. The method of item 17, wherein relative aggressiveness increases as markers from the functional classes are found to vary, in the order of polypeptides for malignant cell transformation, polypeptides for invasion, polypeptides for angiogenesis, and polypeptides for metastasis.
19. The method of any one of items 1-18, further comprising administering an anti-cancer agent when a difference in the level of polypeptides representative of one or more of the functions relative to the reference indicates cancer in the subject.
20. A device for the diagnosis of cancer, the device comprising reagents sufficient to detect, in a sample of platelets or platelet proteins from a subject, the presence and/or amount of a set of polypeptides comprising subsets of polypeptides that are representative of functions in cancer development including each of malignant cell transformation, invasion, angiogenesis and metastasis.
21. The device of item 20, wherein the set of polypeptides comprises a plurality of polypeptides associated with each function.
22. The device of item 20 or item 21, wherein the polypeptides representative of functions in cancer development include cancer stimulatory and cancer inhibitory polypeptides.
23. The device of any one of items 20-22, wherein the polypeptides representative of function in malignant cell transformation include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.
24. The device of any one of items 20-23, wherein the polypeptides representative of functions in malignant cell transformation include two or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.
25. The device of any one of items 20-24, wherein the polypeptides representative of functions in malignant cell transformation include each of RAB1B, RAP1A, Heat shock protein 70 kDa protein 1B, Heat shock 70 kDa protein 6, Heat shock 70 kDa protein 1-like, and Heat shock protein HSP 90-alpha isoform 2.
26. The device of any one of items 20-25, wherein the polypeptides representative of function in cancer cell invasion include one or more of MYCBP2, CD47, FKBP1A, and Tribbles homolog 2.
27. The device of any one of items 20-26, wherein the polypeptides representative of function in cancer cell invasion include two or more of MYCBP2, CD47, FKBP1A, and Tribbles homolog 2.
28. The device of any one of items 20-27, the polypeptides representative of function in angiogenesis include one or more of PF4, TSP-1, basic FGF, VEGF, PDGF beta, and Integrin beta-3.
29. The device of any one of items 20-28, wherein the polypeptides representative of function in angiogenesis include two or more of PF4, TSP-1, basic FGF, VEGF, PDGF beta, and Integrin beta-3.
30. The device of any one of items 20-29, wherein the polypeptides representative of function in angiogenesis include each of PF4, TSP-1, and Integrin beta-3 and optionally one or more of basic FGF, VEGF, and PDGF beta.
31. The device of any one of items 20-30, wherein the polypeptides representative of function in metastasis include one or more of CAMSAP2, SRGN, RANK-L, TGF-beta, IGF-1, and Synaptobrevin homolog YKT6.
32. The device of any one of items 20-31, wherein the polypeptides representative of function in metastasis include two or more of CAMSAP2, SRGN, RANK-L, TGF-beta, IGF-1, and Synaptobrevin homolog YKT6.
33. The device of any one of items 20-32, wherein the polypeptides representative of function in metastasis include each of CAMSAP2, SRGN, and Synaptobrevin homolog YKT6 and optionally one or more of RANK-L, TGF-beta, and/or IGF-1.
34. The device of any one of items 20-33, wherein the device comprises a solid substrate comprising reagents permitting detection of the presence and/or amount of the set of polypeptides.
35. The device of item 26, wherein the solid substrate comprises a lateral flow test strip, a microfluidics chamber, a dipstick, beads, or an enzyme-linked immunosorbent assay (ELISA).
36. The device of item 34 or 35, wherein the solid substrate is coated with a glucosaminoglycan.
37. A kit for the detection, in a sample of platelets or platelet proteins from a subject, of the presence and/or amount of a set of polypeptides comprising subsets of polypeptides that are representative of functions in cancer development including each of transformation, invasion, angiogenesis and metastasis,
   the kit comprising reagents sufficient to detect, in a sample of platelets or platelet proteins from the subject, the presence and/or amount of the set of polypeptides comprising subsets of polypeptides that are representative of functions in cancer development including each of transformation, invasion, angiogenesis and metastasis,
   and packaging materials therefor.
38. A kit for the staging of cancer, the kit comprising reagents necessary for detecting in a platelet sample the presence and/or amount of sets of polypeptides in functional cancer development categories including each of malignant cell transformation, invasion, angiogenesis and metastasis.
39. The kit of item 38, which comprises at least one solid support comprising reagents sufficient to detect the presence and/or amount of the sets of polypeptides.
40. The kit of item 39, wherein the solid support comprises a lateral flow test strip, a microfluidics chamber, a dipstick, beads, or an enzyme-linked immunosorbent assay (ELISA).
41. The kit of item 39 or 40, wherein the solid substrate is coated with a glucosaminoglycan.
42. The kit of any one of items 38-41, wherein the kit comprises reagents for the detection of actin.
43. The kit of any one of items 38-42, wherein the polypeptides representative of function in malignant cell transformation include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.
44. The kit of any one of items 38-43, wherein the polypeptides representative of functions in malignant cell transformation include two or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.
45. The kit of any one of items 38-44, wherein the polypeptides representative of functions in malignant cell transformation include each of RAB1B, RAP1A, Heat shock protein 70 kDa protein 1B, Heat shock 70 kDa protein 6, Heat shock 70 kDa protein 1-like, and Heat shock protein HSP 90-alpha isoform 2.
46. The kit of any one of items 38-45, wherein the polypeptides representative of function in cancer cell invasion include one or more of MYCBP2, CD47, FKBP1A, and Tribbles homolog 2.
47. The kit of any one of items 38-46, wherein the polypeptides representative of function in cancer cell invasion include two or more of MYCBP2, CD47, FKBP1A, and Tribbles homolog 2.
48. The kit of any one of items 38-47, the polypeptides representative of function in angiogenesis include one or more of PF4, TSP-1, basic FGF, VEGF, PDGF beta, and Integrin beta-3.
49. The kit of any one of items 38-48, wherein the polypeptides representative of function in angiogenesis include two or more of PF4, TSP-1, basic FGF, VEGF, PDGF beta, and Integrin beta-3.
50. The kit of any one of items 38-49, wherein the polypeptides representative of function in angiogenesis include each of PF4, TSP-1, and Integrin beta-3 and optionally one or more of basic FGF, VEGF, and PDGF beta.
51. The kit of any one of items 38-50, wherein the polypeptides representative of function in metastasis include one or more of CAMSAP2, SRGN, RANK-L, TGF-beta, IGF-1, and Synaptobrevin homolog YKT6.
52. The kit of any one of items 38-51, wherein the polypeptides representative of function in metastasis include two or more of CAMSAP2, SRGN, RANK-L, TGF-beta, IGF-1, and Synaptobrevin homolog YKT6.
53. The kit of any one of items 38-52, wherein the polypeptides representative of function in metastasis include each of CAMSAP2, SRGN, and Synaptobrevin homolog YKT6 and optionally one or more of RANK-L, TGF-beta, and/or IGF-1.
54. A solid support comprising reagents sufficient to detect, in a sample of platelet proteins from a subject, polypeptides in each of a set of functional cancer development categories including malignant cell transformation, invasion, angiogenesis and metastasis.
55. The solid support of item 54, wherein the reagents sufficient to detect the presence and/or amount of the sets of polypeptides are arranged on the support in four regions, one region each for reagents sufficient to detect polypeptides in functional cancer development categories including malignant cell transformation, invasion, angiogenesis and metastasis, wherein each region comprises reagents sufficient to detect the presence and/or amount of one or more polypeptides in one of the respective functional cancer development categories.
56. The solid support of item 55, wherein each region comprises pooled reagents sufficient to detect the presence of a plurality of polypeptides in the respective functional cancer development category.
57. The solid support of item 55 or 56, wherein the amount of detectable signal in each region upon detection of the sets of polypeptides in a sample of platelets or platelet proteins provides an indication of the presence and/or stage of cancer in the subject from whom the platelets were obtained.
58. A method of diagnosing or staging cancer in a subject, the method comprising contacting a solid support of any one of items 54-57 with a sample of platelets or platelet proteins obtained from the subject, wherein the contacting permits detection of the presence and/or amount of platelet proteins in the sample in one or more of the functional cancer development categories.
59. A kit comprising a solid support of any one of items 54-57 and packaging materials therefor.
60. A method of monitoring cancer treatment for efficacy in a subject, the method comprising:
   (a) measuring, in a first sample of platelets from the subject, the levels of a set of polypeptides comprising subsets of polypeptides that are representative of functions in cancer development including each of malignant cell transformation, invasion, angiogenesis and metastasis;
   (b) measuring, in a second sample of platelets from the subject, taken after administration of a cancer therapy to the subject following step (a), the levels of the set of polypeptides comprising subsets of polypeptides that are representative of functions in cancer development including each of malignant cell transformation, invasion, angiogenesis and metastasis; and
   (c) comparing the levels of the polypeptides measured in steps (a) and (b);
   wherein a change in the level of polypeptides in one or more of the subsets of polypeptides measured in steps (a) and (b) provides an indication of the efficacy of the cancer therapy.
61. The method of item 60, wherein the polypeptides representative of functions in cancer development include cancer stimulatory and cancer inhibitory polypeptides.
62. The method of item 61, wherein a decrease in the level of one or more cancer-stimulatory polypeptides, or an increase in the level of one or more cancer inhibitory polypeptides measured in step (b) relative to those measured in step (a) indicates that the cancer therapy is effective.
63. The method of item 60, wherein a positive prognostic change in the level of polypeptides representative of malignant cell transformation, invasion, angiogenesis or metastasis indicates the therapy is effective, and wherein a negative prognostic change in the level of polypeptides representative of malignant transformation, invasion, angiogenesis or metastasis indicates the therapy is not effective.
64. The method of any one of items 60-63, further comprising:
   when the comparing step indicates the therapy is effective, continuing to administer the therapy at the current dosage and frequency, or reducing the dosage and/or frequency of administration of the therapy; or
   when the comparing step indicates therapy is not effective, administering a different therapy alone or in conjunction with the therapy, or increasing the dosage and/or frequency of the therapy.

The technology is further illustrated by the following example which should not be construed as limiting. The contents of all references cited throughout this application, as well as the figures and table are incorporated herein by reference.

### EXAMPLES

Today people fighting cancers, such as prostate cancer, have limited options for fast, accurate and reliable ways to monitor their disease. For example, current clinical methods to determine if a prostate cancer patient should undergo surgery or if they are responding to chemotherapy, include frequently inaccurate PSA tests, invasive and subjective digital exams, painful tissue biopsies, or simply hoping the patient will outlive the tumor—all of which can significantly stress already suffering patients. Provided herein are methods to efficiently and accurately identify and monitor diseases such as cancer. By evaluating platelets in a small amount of blood (e.g., a teaspoon), the inventors have identified a series of proteins whose presence in platelets strongly indicate particular types of cancer including breast, prostate, and colorectal cancers. Further, the inventors have mapped and measured ‘in-platelet’ or ‘platelet-sequestered’ concentrations of specific proteins and protein combinations for prostate cancer patients. These protein combinations permit a high degree of certainty and predictability as to the stage, speed of evolution and/or regression of the disease.

Tumors can be understood as a “wound that never heals” [1], suggesting that the understanding of wound healing can lead to clues of inhibiting tumor growth. A major component of wound healing in tumor biology are the cell fragments called platelets. Platelets have been shown to be naturally adherent to tumors and to modulate tumor growth. While they act as surveillance systems in tumor dormancy, because platelets of healthy human subjects contain predominantly cancer inhibitors, they can promote tumor growth as cancer progresses The inventors were the first to show platelets actively sequester and concentrate a range of specific tumor angiogenesis-related proteins against a concentration gradient. This sequestration indicates that platelets represent a novel, more accurate, and highly relevant source for the detection of proteins differentially expressed between cancer patients and healthy individuals ([2-4]). However, previous studies into platelet proteins have not characterized the ability of platelets to be utilized as a source of polypeptides, as well as a methodology to diagnose cancer in a precise stage specific manner. Utilizing platelets from healthy individuals, and platelets from prostate cancer patients, through a discovery based mass spectrometry approach, the inventors have identified domains of platelet protein sequestration and novel stage specific prostate polypeptides. The inventors have identified novel domains associated with specific sequestration by platelets including: the heparan sulfate binding domain, the C-type lectin binding domain, the p-selectin binding domain, the elastin binding domain, and the intracellular plexin-D1 binding domain. The inventors have also identified 70 proteins selectively sequestered by platelets to specifically differentiate platelets from healthy subjects from platelets from subjects having Stage T2a prostate cancer. The inventors have also identified 68 proteins selectively sequestered by platelets, which selectively differentiate healthy individuals from stage T3 prostate cancer patients. Finally, the inventors have identified 31 proteins selectively sequestered by platelets which selectively differentiate stage T2 prostate cancer patients from stage T3 prostate cancer patients.

Currently, serum and plasma proteins due to their ease of use are being evaluated as a source and methodology for clinical use polypeptides for the detection and monitoring of cancer [5]. While reliable for late-stage disease, detection, and outcome prediction of large tumors [6];[7], these proteins do not correlate with early tumor growth and, or, tumor invasion [8]. The search for serum or plasma proteins that would be differentially expressed between patients with or without cancer continues. However, plasma and serum analysis has been hindered by the vast number of proteins found within either specimen, and by the large dynamic range over which they are found [9]. Platelets are a-nuclear cell fragments produced by bone marrow megakaryocytes and released directly into the circulation by proplatelet formations. Some platelet-derived proteins, such as platelet factor 4 (PF4) and platelet basic protein (PBP), are synthesized by the megakaryocytes and concentrated in platelets during platelet formation [10-12]. Other proteins such as albumin, IgG, and fibrinogen are synthesized by other cells and taken-up by platelets in the periphery [13-16]. Platelets can be isolated in clinical facilities from whole blood by multiple rounds of centrifugation, with minimal contamination of isolated platelets by plasma proteins or other blood cells, such as erythrocytes or leukocytes. Platelets are easily separated from leukocytes prior to storage or processing, reducing the confounding effects of other cells types. A major difficulty in platelet studies lies in ensuring that the analysis of platelet protein content is performed on resting inactivated platelets, as the release of proteins from activated platelets can be variable. An example is the differential release of VEGF and endostatin upon agonist-induced platelet activation [17]. Under normal physiological conditions, the platelet content of sequestered proteins is quite stable, but the methodology of isolation can impact identity and concentration of sequestered proteins. A number of studies have now catalogued the various proteins present in resting platelets [18-20], yet they have not characterized stage specific platelet proteins in prostate cancer or the use of platelet-sequestered proteins to accurately predict or indicate a particular stage of cancer The active sequestration and release of many angiogenesis-regulatory proteins is likely to play a significant role in angiogenesis and tumor survival [2, 21-25].

The present disclosure provides methods and compositions for use in in the diagnosis, and staging, of precancerous lesions and prostate cancer using platelets selectively sequestered proteins or conditions associated with prostate cancer and/or inflammation in a subject (e.g., a human subject). The methods include isolation of platelet sequestered proteins from subjects (such as by way of isolation from whole blood, platelet rich plasma, plateletpheresis, or differentiation from patient megakaryocytes, or by way of identification in whole blood (e.g., a whole blood kit examining platelet proteins)).

The present disclosure provides methods and compositions for use in in the diagnosis, and staging, of precancerous lesions and prostate cancer using platelet selectively sequestered proteins or conditions associated with prostate cancer and/or inflammation in a subject (e.g., a human subject). In some embodiments, platelet sequestered proteins are quantified by mass spectrometry and/or antibody-based methods, such as, e.g. (ELISA, immunoblotting, flow cytometry), and/or a kit and are utilized to diagnose presence and stage of prostate cancer. Protein concentrations are determined for a desired panel of polypeptides (e.g., those described in Tables 1-3) in platelets from healthy individuals, and individuals who are suspected to have a neoplasia associated with the prostate or another tissue described herein. The protein concentrations can then be normalized to platelet number utilizing a correction factor. The correction factor is obtained from the ratio of platelet counts from a standard range of healthy patients. Deviations greater than 20%, and that are statistically significant utilizing standard statistical methods such as, e.g. (ANOVA, Student's test, Tukey's) are then utilized to determine probability of the presence of prostate cancer, and the clinical stage of prostate cancer.

The inventors identified proteins, which are statistically significantly increased, or decreased, by greater than 20%, within platelets from 5 patients with stage T2 prostate cancer and 5 patients with stage T3 prostate cancer patients compared to platelets form 5 healthy individuals. The inventors have previously shown the ability of platelets to selectively sequester proteins against a concentration gradient to supra-physiological levels. Previous studies have established the presence of angiogenesis regulators in platelets derived from patients with cancer. These previous studies and claims however, have not established mechanisms of sequestration beyond heparan sulfate, identified non-angiogenesis related proteins selectively sequestered by platelets, or established in humans the connection of platelet associated proteins and specific stages of disease. As described in Tables 1, 2 the inventors have identified proteins selectively sequestered into platelets which discriminate healthy individuals from individuals with stage T2 prostate cancer, or individuals with stage T3. They have also identified platelet sequestered proteins which distinguish stage T2 prostate cancer from stage T3 prostate cancer.

Normalization: The inventors normalized platelet associated proteins between healthy individuals and individuals with prostate cancer. Platelet sequestered proteins were isolated, digested, and quantified utilizing mass spectrometry by two different methodologies at two different sites. Only proteins identified by both methodologies and sites were analyzed. Proteins were quantified utilizing a standard “heavy” reference protein at a known concentration. Protein sequence identity was established from a minimum of 3 peptides identified to be protein specific by sequence alignment. Once protein identity was established, platelet protein levels were normalized by a normalization factor. The normalization factor was calculated for each individual patient from the number of platelets in a microliter of blood, and multiplied by a correction factor so each sample contained 200,000 platelets/uL of whole blood. Additional methods of normalization included, but were not limited to, total protein, normalization to albumin, and normalization to a sequestered protein. Only proteins which were significant in multiple methods of normalization were utilized.

Human subjects and preparation of human platelets and plasma: All samples were collected from healthy human volunteers or patients with stage T2 or T3 prostate cancer. The study was approved by the Institutional Review Boards of Tufts Medical Center with study subjects having affirmed written, informed consent to participate in this study. Whole blood was collected into a citrated vacutainer tube and centrifuged at 150 g for 20 minutes to yield platelet rich plasma (PRP). The PRP was centrifuged at 900 g for 10 minutes in order to isolate the platelets from the platelet poor plasma (PPP). Platelet numbers were quantified using a hemocytometer and confirmed by flow cytometry from whole blood and PRP. Isolated platelets and PPP were frozen at −80° C. until further analysis. Samples were obtained from 5 control individuals with no history of cancer and 5 individuals with stage T2a prostate cancer, and patients with stage T3 prostate cancer undergoing surgery due to a clinical diagnosis of prostate cancer. Initial diagnosis was determined through tissue biopsy, with diagnosis of tumor or confirmed by post-surgical pathological examination of resected growth. Platelet counts were performed at the time of blood draw for all patients. No patient samples, tumor or normal, were measured for inflammatory markers. Healthy volunteers had no history of cancer, nor were on any anti-inflammatory drugs.

### Example 1. Platelet Proteins that Change Between Normal and Stage T2 Prostate Cancer

Identical isolated platelets, and platelet poor plasma, normal and patient samples were analyzed by a Waters SCIEX Mass Spectrometer, and a Thermo Orbitrap LC-MS/MS following the suggested manufactures protocol. Only proteins which were not found in plasma or greater, or fewer, than 100% in platelets when compared to plasma were included. The Waters data contained 1369 unique protein IDs and the Thermo Orbitrap data contained 1199 unique protein IDs. A total of 442 proteins that were found by both Waters, and Thermo Orbitrap technologies were utilized for this study. Protein concentrations were normalized as described herein under Normalization. Statistical significance was determined for by either two-tailed student t-test or 2-way ANOVA with a Tukey post hoc test, or repeated measures test (Prism GraphPad Software, La Jolla, CA) to account for multiple comparison testing. Significance was assigned based on p<0.05 for uniformity. Only proteins which were found in all 5 patients, and 20% greater, or fewer, in platelets from cancer patients when compared to platelets from healthy subjects were utilized. Table 1, lists proteins which were different between normal and stage T2a prostate cancer.

Table 2 lists proteins which were different between normal, and stage T3 prostate cancer. Table 3 lists proteins which were different between stage T2, and stage T3 prostate cancer.

The inventors compared the proteins found in plasma to the proteins found in platelets. Only proteins found in platelets and not found in plasma were used. The inventors identified each protein domain found in proteins sequestered by platelets. Only the protein domains found in 5 or more proteins sequestered by platelets were utilized and listed in Table 4.

Quantification of platelet-associated proteins. Due to the proteinaceous characteristic of platelets from cancer subjects, normalization to total protein amount can be problematic. In some embodiments, an alternative method is used to normalize for platelet number. One exemplary method is to equalize the platelet sample using β-actin, a protein with minimal variability between platelets from normal and cancer subjects.

Modifications and variations of the described disclosure will be apparent to those skilled in the art without departing from the scope and spirit of the disclosure. Although the disclosure has been described in connection with specific embodiments, it is understood the disclosure as claimed, should not be unduly limited to such specific embodiments. Modifications of the described modes for carrying out the disclosure, that are obvious to those familiar in the art, are intended to be within the scope of this disclosure. Additional embodiments are listed the claims.

### REFERENCES

1. Dvorak, H. F., Tumors: wounds that do not heal. Similarities between tumor stroma generation and wound healing. N Engl J Med, 1986. 315(26): p. 1650-9.
2. Klement, G. L., et al., Platelets actively sequester angiogenesis regulators. Blood, 2009. 113(12): p. 2835-42.
3. Cervi, D., et al., Platelet-associated PF-4 as a biomarker of early tumor growth. Blood, 2008. 111(3): p. 1201-7.
4. Pietramaggiori, G., et al., Tumors stimulate platelet delivery of angiogenic factors in vivo: an unexpected benefit. Am J Pathol, 2008. 173(6): p. 1609-16.
5. Ludwig, J. A. and J. N. Weinstein, Biomarkers in cancer staging, prognosis and treatment selection. Nat Rev Cancer, 2005. 5(11): p. 845-56.
6. Brattstrom, D., et al., Serum VEGF and bFGF adds prognostic information in patients with normal platelet counts when sampled before, during and after treatment for locally advanced non-small cell lung cancer. Lung Cancer, 2004. 43(1): p. 55-62.
7. Salven, P., et al., High pre-treatment serum level of vascular endothelial growth factor (VEGF) is associated with poor outcome in small-cell lung cancer. Int J Cancer, 1998. 79(2): p. 144-6.
8. Werther, K., I. J. Christensen, and H. J. Nielsen, Prognostic impact of matched preoperative plasma and serum VEGF in patients with primary colorectal carcinoma. Br J Cancer, 2002. 86(3): p. 417-23.
9. Anderson, N. L. and N. G. Anderson, The human plasma proteome: history, character, and diagnostic prospects. Mol Cell Proteomics, 2002. 1(11): p. 845-67.
10. McLaren, K. M. and D. S. Pepper, Immunological localisation of beta-thromboglobulin and platelet factor 4 in human megakaryocytes and platelets. J Clin Pathol, 1982. 35(11): p. 1227-31.
11. Ryo, R., et al., New synthesis of a platelet-specific protein: platelet factor 4 synthesis in a megakaryocyte-enriched rabbit bone marrow culture system. J Cell Biol, 1983. 96(2): p. 515-20.
12. Holt, J. C., et al., Occurrence of platelet basic protein, a precursor of low affinity platelet factor 4 and beta-thromboglobulin, in human platelets and megakaryocytes. Exp Hematol, 1988. 16(4): p. 302-6.
13. Handagama, P. J., et al., Incorporation of a circulating protein into megakaryocyte and platelet granules. Proc Natl Acad Sci USA, 1987. 84(3): p. 861-5.
14. Handagama, P., et al., Endocytosis of fibrinogen into megakaryocyte and platelet alpha-granules is mediated by alpha IIb beta 3 (glycoprotein IIb-IIIa). Blood, 1993. 82(1): p. 135-8.
15. Handagama, P., et al., Platelet alpha-granule fibrinogen, albumin, and immunoglobulin G are not synthesized by rat and mouse megakaryocytes. J Clin Invest, 1990. 86(4): p. 1364-8.
16. Handagama, P. J. and D. F. Bainton, Incorporation of a circulating protein into alpha granules of megakaryocytes. Blood Cells, 1989. 15(1): p. 59-72.
17. Ma, L., et al., Proteinase-activated receptors 1 and 4 counter-regulate endostatin and VEGF release from human platelets. Proc Natl Acad Sci USA, 2005. 102(1): p. 216-20.
18. Moebius, J., et al., The human platelet membrane proteome reveals several new potential membrane proteins. Mol Cell Proteomics, 2005. 4(11): p. 1754-61.
19. Garcia, A., N. Zitzmann, and S. P. Watson, Analyzing the platelet proteome. Semin Thromb Hemost, 2004. 30(4): p. 485-9.
20. Martens, L., et al., The human platelet proteome mapped by peptide-centric proteomics: a functional protein profile. Proteomics, 2005. 5(12): p. 3193-204.
21. Pinedo, H. M., et al., Involvement of platelets in tumour angiogenesis?Lancet, 1998. 352(9142): p. 1775-1777.
22. Verheul, H. M. and H. M. Pinedo, Tumor Growth: A Putative Role for Platelets?Oncologist., 1998. 3(2): p. II.
23. Verheul, H. M., et al., Platelet: transporter of vascular endothelial growth factor. Clin Cancer Res, 1997. 3(12 Pt 1): p. 2187-90.
24. Brill, A., et al., Platelet-derived microparticles induce angiogenesis and stimulate post-ischemic revascularization. Cardiovasc Res, 2005. 67(1): p. 30-8.
25. Brill, A., H. Elinav, and D. Varon, Differential role of platelet granular mediators in angiogenesis. Cardiovasc Res, 2004. 63(2): p. 226-35.
26. Maione, T. E., et al., Inhibition of tumor growth in mice by an analogue of platelet factor 4 that lacks affinity for heparin and retains potent angiostatic activity. Cancer Res, 1991. 51(8): p. 2077-83.
27. Sharpe, R. J., et al., Growth inhibition of murine melanoma and human colon carcinoma by recombinant human platelet factor 4. J Natl Cancer Inst, 1990. 82(10): p. 848-53.
28. Benny, O., et al., In vivo fate and therapeutic efficacy of PF-4/CTF microspheres in an orthotopic human glioblastoma model. FASEB J, 2008. 22(2): p. 488-99.
29. Taylor, S. and J. Folkman, Protamine is an inhibitor of angiogenesis. Nature, 1982. 297(5864): p. 307-12.
30. Aivado, M., et al., Serum proteome profiling detects myelodysplastic syndromes and identifies CXC chemokine ligands 4 and 7 as markers for advanced disease. Proc Natl Acad Sci USA, 2007. 104(4): p. 1307-12.
31. Hanahan, D. and R. A. Weinberg, Hallmarks of cancer: the next generation. Cell, 2011. 144(5): p. 646-74.
32. Hanahan, D. and R. A. Weinberg, The Hallmarks of Cancer. Cell, 2000. 100(1): p. 57-70.

1. A method of diagnosing cancer in a subject, the method comprising:
(a) measuring, in a sample of platelets from the subject, the levels of a set of polypeptides comprising subsets of polypeptides that are representative of functions in cancer development including each of malignant cell transformation, invasion, angiogenesis and metastasis;
(b) comparing the levels of the set of polypeptides measured in step (a) with the levels of the same set of polypeptides in a reference preparation of platelets, plasma, circulating cells or to a reference value or range thereof for each polypeptide;
wherein a difference in the level of polypeptides representative of one or more of the functions relative to the reference is indicative of cancer status in the subject.

2. The method of claim 1, wherein the set of polypeptides comprises a plurality of polypeptides associated with each function.

3. The method of claim 1 or claim 2, wherein the polypeptides representative of functions in cancer development include cancer stimulatory and/or cancer inhibitory polypeptides, and wherein cancer progression is indicated when the balance of cancer stimulatory vs cancer inhibitory polypeptides is disrupted such that cancer stimulatory polypeptides are increased and/or cancer inhibitory polypeptides are decreased relative to the reference.

4. The method of any one of claims 1-3, wherein an increase in one or more cancer stimulatory polypeptides or a decrease in one or more cancer inhibitory polypeptides is indicative of cancer progression.

5. The method of any one of claims 1-3, wherein a decrease in one or more cancer stimulatory polypeptides or an increase in one or more cancer inhibitory polypeptides is indicative of cancer regression or therapy responsiveness.

6. The method of any one of claims 1-5, wherein the method diagnoses prostate cancer, and the polypeptides representative of function in malignant cell transformation include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

7. The method of any one of claims 1-6, wherein the method diagnoses prostate cancer, and the polypeptides representative of functions in malignant cell transformation include two or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

8. The method of any one of claims 1-7, wherein the method diagnoses prostate cancer, and the polypeptides representative of functions in malignant cell transformation include each of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

9. The method of any one of claims 1-8, wherein the method diagnoses prostate cancer, and the polypeptides representative of function in cancer cell invasion include one or more of MYCBP2, CD47, FKBP1A, and Tribbles homolog 2.

10. The method of any one of claims 1-9, wherein the method diagnoses prostate cancer, and the polypeptides representative of function in cancer cell invasion include two or more of MYCBP2, CD47, FKBP1A, and Tribbles homolog 2.

11. The method of any one of claims 1-10, wherein the method diagnoses prostate cancer, and the polypeptides representative of function in angiogenesis include one or more of PF4, TSP-1, basic FGF, VEGF, PDGF beta, and Integrin beta-3.

12. The method of any one of claims 1-11, wherein the method diagnoses prostate cancer, and the polypeptides representative of function in angiogenesis include two or more of PF4, TSP-1, basic FGF, VEGF, PDGF beta, and Integrin beta-3.

13. The method of any one of claims 1-12, wherein the cancer is prostate cancer, and the polypeptides representative of function in angiogenesis include each of PF4, TSP-1, and Integrin beta-3 and optionally basic FGF, VEGF, and/or PDGF beta.

14. The method of any one of claims 1-13, wherein the method diagnoses prostate cancer, and the polypeptides representative of function in metastasis include one or more of CAMSAP2, SRGN, RANK-L, TGF-beta, IGF-1, and Synaptobrevin homolog YKT6.

15. The method of any one of claims 1-14, wherein the method diagnoses prostate cancer, and the polypeptides representative of function in metastasis include two or more of CAMSAP2, SRGN, RANK-L, TGF-beta, IGF-1, and Synaptobrevin homolog YKT6.

16. The method of any one of claims 1-15, wherein the method diagnoses prostate cancer, and the polypeptides representative of function in metastasis include each of CAMSAP2, SRGN, and Synaptobrevin homolog YKT6 and optionally one or more of RANK-L, TGF-beta, or IGF-1.

17. The method of any one of claims 1-16, wherein the aggressiveness or stage of a cancer is indicated by which of the recited functional classes of polypeptides fall within a reference range for subjects having a particular stage of cancer.

18. The method of claim 17, wherein relative aggressiveness increases as markers from the functional classes are found to vary, in the order of polypeptides for malignant cell transformation, polypeptides for invasion, polypeptides for angiogenesis, and polypeptides for metastasis.

19. The method of any one of claims 1-18, further comprising administering an anti-cancer agent when a difference in the level of polypeptides representative of one or more of the functions relative to the reference indicates cancer in the subject.

20. A device for the diagnosis of cancer, the device comprising reagents sufficient to detect, in a sample of platelets or platelet proteins from a subject, the presence and/or amount of a set of polypeptides comprising subsets of polypeptides that are representative of functions in cancer development including each of malignant cell transformation, invasion, angiogenesis and metastasis.

21. The device of claim 20, wherein the set of polypeptides comprises a plurality of polypeptides associated with each function.

22. The device of claim 20 or claim 21, wherein the polypeptides representative of functions in cancer development include cancer stimulatory and cancer inhibitory polypeptides.

23. The device of any one of claims 20-22, wherein the polypeptides representative of function in malignant cell transformation include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

24. The device of any one of claims 20-23, wherein the polypeptides representative of functions in malignant cell transformation include two or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

25. The device of any one of claims 20-24, wherein the polypeptides representative of functions in malignant cell transformation include each of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

26. The device of any one of claims 20-25, wherein the polypeptides representative of function in cancer cell invasion include one or more of MYCBP2, CD47, FKBP1A, and Tribbles homolog 2.

27. The device of any one of claims 20-26, wherein the polypeptides representative of function in cancer cell invasion include two or more of MYCBP2, CD47, FKBP1A, and Tribbles homolog 2.

28. The device of any one of claims 20-27, the polypeptides representative of function in angiogenesis include one or more of PF4, TSP-1, basic FGF, VEGF, PDGF beta, and Integrin beta-3.

29. The device of any one of claims 20-28, wherein the polypeptides representative of function in angiogenesis include two or more of PF4, TSP-1, basic FGF, VEGF, PDGF beta, and Integrin beta-3.

30. The device of any one of claims 20-29, wherein the polypeptides representative of function in angiogenesis include each of PF4, TSP-1, and Integrin beta-3 and optionally one or more of basic FGF, VEGF, and PDGF beta.

31. The device of any one of claims 20-30, wherein the polypeptides representative of function in metastasis include one or more of CAMSAP2, SRGN, RANK-L, TGF-beta, IGF-1, and Synaptobrevin homolog YKT6.

32. The device of any one of claims 20-31, wherein the polypeptides representative of function in metastasis include two or more of CAMSAP2, SRGN, RANK-L, TGF-beta, IGF-1, and Synaptobrevin homolog YKT6.

33. The device of any one of claims 20-32, wherein the polypeptides representative of function in metastasis include each of CAMSAP2, SRGN, and Synaptobrevin homolog YKT6 and optionally one or more of RANK-L, TGF-beta, and/or IGF-1.

34. The device of any one of claims 20-33, wherein the device comprises a solid substrate comprising reagents permitting detection of the presence and/or amount of the set of polypeptides.

35. The device of claim 26, wherein the solid substrate comprises a lateral flow test strip, a microfluidics chamber, a dipstick, beads, or an enzyme-linked immunosorbent assay (ELISA).

36. The device of claim 34 or 35, wherein the solid substrate is coated with a glucosaminoglycan.

37. A kit for the detection, in a sample of platelets or platelet proteins from a subject, of the presence and/or amount of a set of polypeptides comprising subsets of polypeptides that are representative of functions in cancer development including each of transformation, invasion, angiogenesis and metastasis,
the kit comprising reagents sufficient to detect, in a sample of platelets or platelet proteins from the subject, the presence and/or amount of the set of polypeptides comprising subsets of polypeptides that are representative of functions in cancer development including each of transformation, invasion, angiogenesis and metastasis,
and packaging materials therefor.

38. A kit for the staging of cancer, the kit comprising reagents necessary for detecting in a platelet sample the presence and/or amount of sets of polypeptides in functional cancer development categories including each of malignant cell transformation, invasion, angiogenesis and metastasis.

39. The kit of claim 38, which comprises at least one solid support comprising reagents sufficient to detect the presence and/or amount of the sets of polypeptides.

40. The kit of claim 39, wherein the solid support comprises a lateral flow test strip, a microfluidics chamber, a dipstick, beads, or an enzyme-linked immunosorbent assay (ELISA).

41. The kit of claim 39 or 40, wherein the solid substrate is coated with a glucosaminoglycan.

42. The kit of any one of claims 38-41, wherein the kit comprises reagents for the detection of actin.

43. The kit of any one of claims 38-42, wherein the polypeptides representative of function in malignant cell transformation include one or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

44. The kit of any one of claims 38-43, wherein the polypeptides representative of functions in malignant cell transformation include two or more of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

45. The kit of any one of claims 38-44, wherein the polypeptides representative of functions in malignant cell transformation include each of RAB1B, RAP1A, Heat shock protein 70 kDa protein 1B, Heat shock 70 kDa protein 6, Heat shock 70 kDa protein 1-like, and Heat shock protein HSP 90-alpha isoform 2.

46. The kit of any one of claims 38-45, wherein the polypeptides representative of function in cancer cell invasion include one or more of MYCBP2, CD47, FKBP1A, and Tribbles homolog 2.

47. The kit of any one of claims 38-46, wherein the polypeptides representative of function in cancer cell invasion include two or more of MYCBP2, CD47, FKBP1A, and Tribbles homolog 2.

48. The kit of any one of claims 38-47, the polypeptides representative of function in angiogenesis include one or more of PF4, TSP-1, basic FGF, VEGF, PDGF beta, and Integrin beta-3.

49. The kit of any one of claims 38-48, wherein the polypeptides representative of function in angiogenesis include two or more of PF4, TSP-1, basic FGF, VEGF, PDGF beta, and Integrin beta-3.

50. The kit of any one of claims 38-49, wherein the polypeptides representative of function in angiogenesis include each of PF4, TSP-1, and Integrin beta-3 and optionally one or more of basic FGF, VEGF, and PDGF beta.

51. The kit of any one of claims 38-50, wherein the polypeptides representative of function in metastasis include one or more of CAMSAP2, SRGN, RANK-L, TGF-beta, IGF-1, and Synaptobrevin homolog YKT6.

52. The kit of any one of claims 38-51, wherein the polypeptides representative of function in metastasis include two or more of CAMSAP2, SRGN, RANK-L, TGF-beta, IGF-1, and Synaptobrevin homolog YKT6.

53. The kit of any one of claims 38-52, wherein the polypeptides representative of function in metastasis include each of CAMSAP2, SRGN, and Synaptobrevin homolog YKT6 and optionally one or more of RANK-L, TGF-beta, and/or IGF-1.

54. A solid support comprising reagents sufficient to detect, in a sample of platelet proteins from a subject, polypeptides in each of a set of functional cancer development categories including malignant cell transformation, invasion, angiogenesis and metastasis.

55. The solid support of claim 54, wherein the reagents sufficient to detect the presence and/or amount of the sets of polypeptides are arranged on the support in four regions, one region each for reagents sufficient to detect polypeptides in functional cancer development categories including malignant cell transformation, invasion, angiogenesis and metastasis, wherein each region comprises reagents sufficient to detect the presence and/or amount of one or more polypeptides in one of the respective functional cancer development categories.

56. The solid support of claim 55, wherein each region comprises pooled reagents sufficient to detect the presence of a plurality of polypeptides in the respective functional cancer development category.

57. The solid support of claim 55 or 56, wherein the amount of detectable signal in each region upon detection of the sets of polypeptides in a sample of platelets or platelet proteins provides an indication of the presence and/or stage of cancer in the subject from whom the platelets were obtained.

58. A method of diagnosing or staging cancer in a subject, the method comprising contacting a solid support of any one of claims 54-57 with a sample of platelets or platelet proteins obtained from the subject, wherein the contacting permits detection of the presence and/or amount of platelet proteins in the sample in one or more of the functional cancer development categories.

59. A kit comprising a solid support of any one of claims 54-57 and packaging materials therefor.

60. A method of monitoring cancer treatment for efficacy in a subject, the method comprising:
(a) measuring, in a first sample of platelets from the subject, the levels of a set of polypeptides comprising subsets of polypeptides that are representative of functions in cancer development including each of malignant cell transformation, invasion, angiogenesis and metastasis;
(b) measuring, in a second sample of platelets from the subject, taken after administration of a cancer therapy to the subject following step (a), the levels of the set of polypeptides comprising subsets of polypeptides that are representative of functions in cancer development including each of malignant cell transformation, invasion, angiogenesis and metastasis; and
(c) comparing the levels of the polypeptides measured in steps (a) and (b);
wherein a change in the level of polypeptides in one or more of the subsets of polypeptides measured in steps (a) and (b) provides an indication of the efficacy of the cancer therapy.

61. The method of claim 60, wherein the polypeptides representative of functions in cancer development include cancer stimulatory and cancer inhibitory polypeptides.

62. The method of claim 61, wherein a decrease in the level of one or more cancer-stimulatory polypeptides, or an increase in the level of one or more cancer inhibitory polypeptides measured in step (b) relative to those measured in step (a) indicates that the cancer therapy is effective.

63. The method of claim 60, wherein a positive prognostic change in the level of polypeptides representative of malignant cell transformation, invasion, angiogenesis or metastasis indicates the therapy is effective, and wherein a negative prognostic change in the level of polypeptides representative of malignant transformation, invasion, angiogenesis or metastasis indicates the therapy is not effective.

64. The method of any one of claims 60-63, further comprising:
when the comparing step indicates the therapy is effective, continuing to administer the therapy at the current dosage and frequency, or reducing the dosage and/or frequency of administration of the therapy; or
when the comparing step indicates therapy is not effective, administering a different therapy alone or in conjunction with the therapy, or increasing the dosage and/or frequency of the therapy.
